(19) United States
(12) Patent Application Publication
(10) Pub. No.: US 2011/0143380 A1
(43) Pub. Date: Jun. 16, 2011

(54) ALZHEIMER'S DISEASE BIOMARKERS AND METHODS OF USE

(75) Inventors: David Holtzman, St. Louis, MO (US); Yan Hu, St. Louis, MO (US); Robert Reid Townsend, St. Louis, MO (US); Richard J. Perrin, (US); Anne Fagan Niven, (US); Rebecca Craig-Schapiro, (US)

(73) Assignee: THE WASHINGTON UNIVERSITY, St. Louis, MO (US)

(21) Appl. No.: 12/954,259

(22) Filed: Nov. 24, 2010

Related U.S. Application Data
(63) Continuation-in-part of application No. 12/282,799, filed on Feb. 18, 2009, now abandoned.
(60) Provisional application No. 60/782,175, filed on Mar. 14, 2006.

(51) Int. Cl. G01N 33/53; C07K 2/00

(52) U.S. Cl. 435/7.92; 530/350

(57) ABSTRACT

The invention encompasses biomarkers for AD, a method for detecting AD, a method of monitoring AD, and a kit for quantifying biomarkers for AD.

### RELATED APPLICATIONS

The present application is a continuation-in-part of U.S. patent application Ser. No. 12/282,799, filed Feb. 18, 2009, which claims priority to PCT/US07/63142, filed Mar. 12, 2007, which claim priority to U.S. Provisional Application 60/782,175, filed Mar. 14, 2006, each of which is hereby incorporated by reference in their entirety.

### GOVERNMENTAL RIGHTS

This invention was made with government support under AG025662 awarded by the NIH. The government has certain rights in the invention.

### FIELD OF THE INVENTION

The invention generally relates to biomarkers for Alzheimer's disease, methods of detecting Alzheimer's disease, methods of monitoring Alzheimer's disease, and kits for detecting biomarkers for Alzheimer's disease.

### BACKGROUND OF THE INVENTION

Alzheimer's disease (AD) will likely become the greatest public health crisis in the United States within the next 2-3 decades if left unchecked. There are currently no proven treatments that delay the onset or prevent the progression of AD, although a few promising candidates are being developed. During the development of these therapies, it will be very important to have biomarkers that can identify individuals at high risk for AD or at the earliest clinical stage of AD in order to target them for therapeutic trials, disease-modifying therapies and to monitor their therapy. Clinicopathological studies suggest that AD pathology (particularly the buildup of amyloid plaques) begins 10-20 years before cognitive symptoms. Even the earliest clinical symptoms of AD are accompanied by, and likely due to, neuronal/synaptic dysfunction and/or cell death. Thus, it will be critical to identify individuals with “preclinical” and very early stage AD, prior to marked clinical symptoms and neuronal loss, so new therapies will have the greatest clinical impact.

A definitive diagnosis of AD can still only be obtained via neuropathologic evaluation at autopsy. Investigators at the Washington University School of Medicine (WUSM) developed a Clinical Dementia Rating (CDR) scale in which an individual's cognition is rated as normal (CDR 0), or demented with severities of very mild, mild, moderate or severe (CDR 0.5, 1, 2 or 3, respectively) (See Morris, Neurology, 1993; 43:2412, hereby included by reference). Individuals diagnosed with possible/probable dementia of the Alzheimer's type (DAT) are usually CDR 1 or greater. One challenge has been to diagnose individuals at earlier stages, when clinical symptoms are less severe. During these early stages (CDR 0.5, often lasting 2-5 years or longer), the majority of individuals meet clinical criteria for mild cognitive impairment (MCI) (Peterson et al., Arch. Neurol, 1999; 56:303). Data suggest an early and insidious pathogenesis of AD, the clinical manifestation of which becomes apparent only after substantial neuronal cell death and synapse loss has taken place. These findings have profound implications for AD therapeutic and diagnostic strategies.

At present, a few AD biomarkers have been identified that may differentiate individuals with clinical disease (i.e., DAT) from those who are cognitively normal. Mean cerebral spinal fluid (CSF) amyloid beta (Aβ42) levels have been consistently reported to be decreased in AD, including cases of mild dementia, although this decrease may not be specific for AD. CSF Aβ42 is also decreased in MCI, but there is great overlap with control group values. Many studies have reported elevated levels of CSF total tau (and phosphorylated forms) in AD patients. However, similar to Aβ42, there is significant overlap between individual tau values in MCI/AD and control groups, and this increase is not specific for AD. In addition to Aβ42 and tau, differences in other candidate AD biomarkers that likely reflect CNS damage have been observed, including isoprostanes, and 4-hydroxy-2-nonenal (markers of oxidative damage), and sulfatide, a sphingolipid produced by oligodendrocytes. To date, however, none of these individual candidate markers have achieved levels of sensitivity and specificity acceptable for use in disease diagnosis.

### SUMMARY OF THE INVENTION

One aspect of the invention encompasses a biomarker for AD. The biomarker comprises the level of YKL-40 in a bodily fluid of a subject.

Another aspect of the invention encompasses a biomarker for AD. The biomarker comprises the level of CSF YKL-40/Aβ42 in a sample from a subject.

Yet another aspect of the invention encompasses a method for detecting or monitoring AD. Generally speaking, the method comprises quantifying the level of YKL-40 in a bodily fluid of the subject and determining if the quantified level of YKL-40 is elevated in comparison to the average YKL-40 level for a subject with a CDR of 0.

Still another aspect of the invention encompasses a kit for quantifying YKL-40 in a bodily fluid of a subject. The kit comprises the means to quantify YKL-40 and instructions.

Other aspects and iterations of the invention are described in more detail below.

### BRIEF DESCRIPTION OF THE FIGURES

FIG. 1 depicts the 2-D DIGE analysis of CSF prior to and following depletion of high abundance proteins. The same amount of protein (19 micrograms) in CSF prior to depletion (A) and following depletion (B) and in the retained proteins (C) was labeled with Cy2 (blue), Cy3 (green), and Cy5 (red), respectively, and analyzed on a single gel (10% isocratic SDS-PAGE gel). (D) overlay of all three fluorescent images demonstrates the position of the depleted proteins (pink) with respect to the low abundance proteins revealed by the depletion method.

FIG. 2 depicts a representative 2-D DIGE image (Cy2-labeled) of CSF that has been depleted of six high abundance proteins. 50 micrograms of protein was labeled and resolved first on a pH 3-10 IPG strip and further separated on a 10-20% gradient SDS-PAGE gel.

FIG. 3 depicts representative gel images and 3-D representations of one of the apoE spots that displayed intraindividual variation. Shown here are the data from Subject 2. There is a 3.1-fold change of the levels of this apoE spot between the two time points. (A) represents timepoint 1; (B) represents timepoint 2.

FIG. 4 depicts the hierarchical clustering of the 2-D DIGE profiles of 306 matched proteins spots from the 12 CSF samples from six individual subjects at time 1 (T1) and 2 weeks (T2). Each 2-D DIGE profile (column) contains 306 matched protein spots. Lines correspond to individual proteins, and colors represent their standard abundance after a log transformation and Z-score normalization (red, more abundant; green, less abundant). The CDR 0.5 samples are marked with an asterisk. Spotfire software was used to generate the cluster tree and the heat map. Distance in the cluster tree depicted here is not a reflection of similarity or strength of association.

FIG. 5 depicts the multidimensional scaling analysis of the 2-D DIGE profiles of 12 CSF samples from six individual subjects at time 1 (T1) and then 2 weeks later (T2). A 2-D projection of the 3-D scatter plot is shown. The proteomic profile of each sample is represented by a point. The axes correspond to the first three principal components. A single color has been used to label two intraindividual CSF samples.

FIG. 6 depicts graphs showing the CSF levels of biomarkers in CDR group of 0, 0.5, and 1. The graphs were generated using unadjusted raw data. One-way ANOVA analysis was performed to compare the average levels of candidate biomarkers in the three groups and where overall p<0.05, Bonferroni's multiple comparison test was done to examine which comparisons generate statistically significant differences (denoted by asterisks). (A) ACT; (B) ATIII; (C) ZAG; (D) CDNP1.

FIG. 7 depicts graphs showing that the mean levels of CSF Aβ₄₂ are decreased (A) and levels of total tau are increased (B) in very mild AD vs. control subjects. Clinical dementia rating (CDR) 0 equals no cognitive impairment, CDR 0.5 represents very mild dementia, and CDR 1 represents mild dementia due to AD. P values calculated using the raw data (P) and those calculated using the log-transformed and adjusted dataset (P*) are also displayed.

FIG. 8 depicts graphs showing the levels of selected candidate biomarkers in a large CSF sample set as assayed by ELISA. P values calculated using the raw data (P) and those calculated using the log-transformed and adjusted dataset (P*) are displayed. ACT, ATIII, and ZAG are significantly increased in AD (CDR 0.5 and 1) vs. control (CDR 0) samples. (A) ACT; (B) ZAG; (C) Gelsolin; (D) ATIII; (E) CDNP1; (F) AGT.

FIG. 9 depicts graphs showing that the levels of ACT, ATIII, and ZAG are not significantly different in plasma between AD (CDR 0.5 and 1) vs. control (CDR 0) samples. (A) ACT; (B) ATIII; (C) ZAG

FIG. 10 depicts graphs showing the correlations between the CSF and plasma levels of candidate biomarkers, including ACT (A), ATIII (B), and ZAG (C).

FIG. 11 depicts a graph showing the receiver operating characteristic curve (ROC) for the normalized and adjusted CSF concentrations of each biomarker candidate and the optimum linear combination (Optimum) combining data from all biomarkers.

FIG. 12 depicts that YKL-40 appeared in four gel features that were more abundant in the CDR1 group. (A) A representative 2-D DIGE image of CSF from the discovery cohort. Samples were depleted of six highly abundant proteins, fluorescently labeled, and subjected to isoelectric focusing followed by SDS-PAGE. YKL-40 is more abundant in four spots in the CDR 1 group, labeled 1-4 in the inset, with mean fold changes of 1.41, 1.50, 1.46, 1.32, respectively. (B) Sequence coverage of human YKL-40 by mass spectrometry. Indicated in red is the compilation of peptides identified in the four spots, The signal sequence is shown in green, and polymorphisms are indicated by boxes. This sequence is a full-length chitinase 3-like 1 protein.

FIG. 13 depicts that mean YKL-40 is increased in the CSF of CDR 0.5 and CDR 1 subjects by ELISA, and the degree of overlap between clinical groups is comparable for all biomarkers evaluated. (A) CSF YKL-40 was significantly higher in the CDR 1 group as compared to the CDR 0 group (p=0.0016, unpaired student's t-test): CDR 0=293.6+/−23.9; CDR 1=422.2+/−30.0, ng/mL. (B) CSF from a larger, independent sample set (N=292) was analyzed for YKL-40. Mean CSF YKL-40 was significantly higher in the CDR 0.5 and CDR 1 groups as compared to the CDR 0 group (**p=0.004, ***p<0.0001, One-way ANOVA with Welch's correction for unequal variances, Tukey post-hoc Test) (CDR 0=282.1+/−6.7; CDR 0.5=358.9+/−16.9; CDR 1=351.7+/−22.6, 468433.1 ng/mL, mean+/−SEM). (C) Mean CSF YKL-40/Aβ42 was significantly higher in the CDR 0.5 and CDR 1 groups as compared to the CDR 0 group (***p<0.0001, One-way ANOVA with Welch's correction for unequal variances, Tukey post-hocTest). (D) Mean CSF Aβ42 was significantly higher while (E) Mean CSF tau was significantly lower in the CDR 0.5 and CDR 1 groups as compared to the CDR 0 group (***p<0.0001, Oneway ANOVA with Welch's correction for unequal variances, Tukey post-hoc Test).

FIG. 14 depicts that CSF YKL-40 is increased in FTLD and decreased in PSP as shown by ELISA. (A) CSF samples from subjects with FTLD and PSP were analyzed for YKL-40, and levels were compared to those of the validation cohort (CDR 0 and CDR>0, N=292). Analyses were adjusted for age. CSF YKL-40 was significantly higher in the FTLD group as compared to the PSP, CDR 0, and CDR>0 groups (***p<0.0001, ANCOVA, LSD post-hoc Test). CSF YKL-40 levels trended lower in the PSP group as compared to the CDR>0 group. (B-C) CSF YKL-40 and CSF tau values correlated strongly in the FTLD group, but did not correlate in the PSP group.

FIG. 15 depicts that in the validation cohort, CSF YKL-40 levels do not vary based on gender and are not correlated with CSF Aβ42. However, CSF YKL-40 levels are correlated with age, CSF tau, CSF p-tau181, and mean cortical PIB binding potential.

FIG. 16 depicts CSF YKL-40/Aβ42, tau/Aβ42, and p-tau/Aβ42 as predictors of (A) conversion from CDR 0 to CDR>0 and (B) progression from CDR 0.5 to CDR>0.5. Rates of conversion and progression are shown with red curves representing the upper tertile and black curves representing the lower two tertiles. The bottom panel shows for the CSF YKL-40/Aβ42 analyses the number of subjects in the upper and lower tertiles at baseline and at each year of follow-up.

FIG. 17 depicts a graph showing that the Cox proportional hazards models were used to assess the ability of CSF YKL-40/Aβ42, tau/Aβ42, and ptau/Aβ42 to predict conversion from cognitive normalcy (CDR 0) to cognitive impairment (CDR>0) (top) and progression from very mild dementia (CDR 0.5) to mild or moderate dementia (CDR>0.5) (bottom). HR, hazard ratio; CI, confidence interval.

FIG. 18 depicts that CSF YKL-40, tau, p-tau, and Aβ42 as predictors of conversion from CDR 0 to CDR>0. Rates of conversion are shown with red curves representing the upper tertile and black curves representing the lower two tertiles.

FIG. 19 depicts that the plasma samples of the validation cohort (N=237) were evaluated for YKL-40 by ELISA. (A) Mean plasma YKL-40 was significantly higher in the CDR 0.5 and CDR 1 groups as compared to the CDR 0 group (+p=0.046, *p=0.031, One-way ANOVA, Tukey post-hoc Test) (CDR 0=62.5+/−3.4; CDR 0.5=81.1+/−8.0; CDR 1=91.9+/−15.0, ng/mL, mean+/−SEM). (B) CSF and plasma YKL-40 levels are significantly correlated (r=0.2376, p=0.0002).

FIG. 20 depicts that Plasma YKL-40 levels do not vary based on gender, but are correlated with age. Plasma YKL-40 levels are not correlated with other CSF biomarkers such as Aβ42, tau, ptau181, or with mean cortical PIB binding potential.

FIG. 21 depicts that in AD neocortex, YKL-40 immunoreactivity is observed in the vicinity of thioflavin Spositive fibrillar amyloid plaques (A,B,C). YKL-40 immunoreactivity is present within a subset of GFAP-positive astrocytes (D) and not in LN-3-positive microglia (E,F). YKL-40 is also observed in cell processes associated with plaques (G); these processes lack reactivity for dystrophic neurite marker PHF-1 (H,I) and microglial marker LN-3 (J,K,L representing adjacent focal planes), and may represent astrocytic processes. YKL-40 immunoreactivity is also observed in occasional neurons in the superficial white matter (M,N,O), some of which contain neurofibrillary tangles (evidenced by PHF-1 staining, N,O). Scale bars=50 μm; scale bar in A applies to A-C; scale bar in D applies to D-O, with the exception of N.

### DETAILED DESCRIPTION OF THE PREFERRED EMBODIMENTS

Given the early, insidious pathogenesis of AD, combined with the theory that neuronal degeneration is easier to slow or halt than to reverse, it is vital to identify biomarkers that can detect the disease before or during the early development of symptoms and irreversible pathologic damage. Such biomarkers could be used for AD screening and diagnosis, as well as potentially for assessing response to new therapies. Despite the existence of a few promising CSF biomarkers for early stage AD as described above, these candidate markers have not fulfilled the consensus criteria necessary for use in individual diagnosis. Given the probability of multiple underlying pathogenic mechanisms of late-onset AD, it is likely that a battery of biomarkers will be more useful than an individual marker. Therefore, new and better biomarkers must be identified.

To this end, the present invention provides novel AD biomarkers present in the bodily fluid of a subject. The level of these biomarkers correlate with CDR score, and therefore may allow a more accurate diagnosis or prognosis of AD in subjects that are at risk for AD, that show no clinical signs of AD, or that show minor clinical signs of AD. Furthermore, the biomarkers may allow the monitoring of AD, such that a comparison of biomarker levels allows an evaluation of disease progression in subjects that have been diagnosed with AD, or that do not yet show any clinical signs of AD. Moreover, the AD biomarkers of the invention may be used in concert with known AD biomarkers such that a more accurate diagnosis or prognosis of AD may be made.

### I. Biomarkers to Detect Alzheimer's Disease

One aspect of the present invention provides biomarkers to detect AD. A biomarker is typically a protein, found in a bodily fluid, whose level varies with disease state and may be readily quantified. The quantified level may then be compared to a known value. The comparison may be used for several different purposes, including but not limited to, diagnosis of AD, prognosis of AD, and monitoring treatment of AD.

Through proteomic screening performed as detailed in the examples, several novel biomarkers have been identified for AD. In one embodiment, the level of a serine protease inhibitor is a biomarker for AD. Examples of serine protease inhibitors include alpha 1-antitrypsin, alpha 1-antichymotrypsin, alpha 2-antiplasmin, antithrombin III, complement 1-inhibitor, neuroserpin, plasminogen activator inhibitor-1 and 2, and protein Z-related protease inhibitor (ZPI). In another embodiment the biomarker is the level of al-antichymotrypsin (ACT). In yet another embodiment, the biomarker is the level of antithrombin III (ATM). In an alternative embodiment, the biomarker is the level of zinc-alpha-2-glycoprotein (ZAG). In another alternative embodiment, the biomarker is the level of carnosinase 1 (CNDP1). Still in another embodiment, the biomarker is the level of chitinase-3 like-1 (YKL-40).

Each of the biomarkers identified above may be used in concert with another biomarker for purposes including but not limited to diagnosis of AD, prognosis of AD, and monitoring treatment of AD. For instance, two or more, three or more, four or more, five or more, or six or more AD biomarkers may be used in concert. As explained above, there are several known biomarkers for AD. In one embodiment, two or more biomarkers from the group comprising ACT, ATIII, ZAG, CNDP1, Aβ42, YKL-40 and tau are used in concert. In yet another embodiment, three or more biomarkers from the group comprising ACT, ATIII, ZAG, CNDP1, Aβ42, YKL-40 and tau are used in concert. In still another embodiment, four or more biomarkers from the group comprising ACT, ATIII, ZAG, CNDP1, Aβ42, YKL-40 and tau are used in concert. In another alternative embodiment, five or more biomarkers from the group comprising ACT, ATIII, ZAG, CNDP1, Aβ42, YKL-40 and tau are used in concert. In yet still another embodiment, ACT, ATIII, ZAG, CNDP1, Aβ42, YKL-40 and tau are used in concert as biomarkers for AD.

a. Bodily Fluids

The levels of AD biomarkers of the invention may be quantified in several different bodily fluids. Non-limiting examples of bodily fluid include whole blood, plasma, serum, bile, lymph, pleural fluid, semen, saliva, sweat, urine, and CSF. In one embodiment, the bodily fluid is selected from the group comprising whole blood, plasma, and serum. In another embodiment, the bodily fluid is whole blood. In yet another embodiment, the bodily fluid is plasma. In still yet another embodiment, the bodily fluid is serum. In an exemplary embodiment, the bodily fluid is CSF.

As will be appreciated by a skilled artisan, the method of collecting a bodily fluid from a subject can and will vary depending upon the nature of the bodily fluid. Any of a variety of methods generally known in the art may be utilized to collect a bodily fluid from a subject. Generally speaking, the method preferably maintains the integrity of the AD biomarker such that it can be accurately quantified in the bodily fluid. One method of collecting CSF is detailed in the examples. Methods for collecting blood or fractions thereof are well known in the art. For example, see U.S. Pat. No. 5,286,262, which is hereby incorporated by reference in its entirety.

A bodily fluid may be tested from any mammal known to suffer from Alzheimer's disease or used as a disease model for Alzheimer's disease. In one embodiment, the subject is a rodent. Examples of rodents include mice, rats, and guinea pigs. In another embodiment, the subject is a primate. Examples of primates include monkeys, apes, and humans. In an exemplary embodiment, the subject is a human. In some embodiments, the subject has no clinical signs of AD. In other embodiments, the subject has mild clinical signs of AD, for instance, corresponding to a CDR score of 0.5. In yet other embodiments, the subject may be at risk for AD. In still other embodiments, the subject has been diagnosed with AD.

b. Level of Biomarker

The level of the biomarker may encompass the level of protein concentration or the level of enzymatic activity. In either embodiment, the level is quantified, such that a value, an average value, or a range of values is determined. In one embodiment, the level of protein concentration of the AD biomarker is quantified. In another embodiment, the concentration of ATIII is quantified. In yet another embodiment, the concentration of ACT is quantified. In still another embodiment, the concentration of ZAG is quantified. In still yet another embodiment, the concentration of CNDP1 is quantified. In another alternative embodiment, the concentration of YKL-40 is quantified.

There are numerous known methods and kits for measuring the amount or concentration of a protein in a sample, including ELISA, western blot, absorption measurement, colorimetric determination, Lowry assay, Bicinchoninic acid assay, or a Bradford assay. Commercial kits include ProteoQwest™ Colorimetric Western Blotting Kits (Sigma-Aldrich, Co.), QuantiPro™ bicinchoninic acid (BCA) Protein Assay Kit (Sigma-Aldrich, Co.), FluoroProfile™ Protein Quantification Kit (Sigma-Aldrich, Co.), the Coomassie Plus—The Better Bradford Assay (Pierce Biotechnology, Inc.), and the Modified Lowry Protein Assay Kit (Pierce Biotechnology, Inc.). In certain embodiments, the protein concentration is measured by ELISA. For instance, the level of ATIII or YKL-40 may be quantified by ELISA as described in the examples.

In another embodiment, the level of enzymatic activity of the biomarker is quantified. Generally, enzyme activity may be measured by means known in the art, such as measurement of product formation, substrate degradation, or substrate concentration, at a selected point(s) or time(s) in the enzymatic reaction. In one embodiment, the enzyme activity of ATIII is quantified. In another embodiment, the enzyme activity of ACT is quantified. In yet another embodiment, the enzyme activity of CNDP1 is quantified. In another alternative embodiment, the enzyme activity of YKL-40 is quantified. There are numerous known methods and kits for measuring enzyme activity. For example, see U.S. Pat. No. 5,654,152. Some methods may require purification of the AD biomarker prior to measuring the enzymatic activity of the biomarker. A pure biomarker constitutes at least about 90%, preferably, 95% and even more preferably, at least about 99% by weight of the total protein in a given sample. AD biomarkers of the invention may be purified according to methods known in the art, including, but not limited to, ion-exchange chromatography, size-exclusion chromatography, affinity chromatography, differential solubility, differential centrifugation, and HPLC. (See Current Protocols in Molecular Biology, Eds. Ausubel, et al., Greene Publ. Assoc., Wiley-Interscience, New York)

### II. Methods of Using the Biomarkers

a. Using Biomarkers for the Diagnosis or Prognosis of AD

In one embodiment, the invention encompasses a method for detecting AD comprising quantifying the level of an AD biomarker in a bodily fluid of a subject and subsequently determining if the quantified level of the biomarker is elevated or depressed in comparison to the average level of the biomarker for a subject with a CDR of 0. The subject may have no clinical signs of AD, the subject might be at risk for AD, or alternatively, the subject might show mild dementia (CDR of 0.5). The average level of the biomarker for a subject with a CDR of 0 refers to the arithmetic average of the biomarker level in a bodily fluid of at least 50 subjects with a CDR of 0.

An elevated or depressed biomarker level may lead to either a diagnosis or prognosis of AD. In one embodiment, an elevated biomarker level indicates a diagnosis of AD. In another embodiment, an elevated biomarker level indicates a prognosis of AD. In yet another embodiment, a depressed biomarker level indicates a diagnosis of AD. In still yet another embodiment, a depressed biomarker level indicates a prognosis of AD.

A skilled artisan will realize that whether an elevated or a depressed biomarker level in comparison to the average level of the biomarker for a subject with a CDR of 0 is indicative of AD will depend on the biomarker in question. In one embodiment, an elevated level of ATIII indicates a diagnosis or prognosis of AD. In another embodiment, an elevated level of ACT indicates a diagnosis or prognosis of AD. In yet another embodiment, an elevated level of tau indicates a diagnosis or prognosis of AD. In still another embodiment, a depressed level of Aβ42 indicates a diagnosis or prognosis of AD. In yet still another embodiment, a modulated level of ZAG indicates a diagnosis or prognosis of AD. In an alternative embodiment, a depressed level of CNDP1 indicates a diagnosis or prognosis of AD. Yet in another alternative embodiment, an elevated level of YKL-40 indicates a diagnosis or prognosis of AD.

An AD biomarker of the invention may be quantified in concert with another known AD biomarker as detailed in Part I above. For example, ATIII may be used as an AD biomarker in concert with Aβ42. In this example, a simultaneously elevated ATIII level and a depressed Aβ42 level in a bodily fluid of a subject would be indicative of a diagnosis or prognosis of AD. For another instance, YKL-40 may be used as an AD biomarker in concert with Aβ42, such that an elevated YKL-40/Aβ42 ratio in a bodily fluid of a subject would be indicative of a diagnosis or prognosis of AD.

The percent elevation or depression of an AD biomarker compared to the average level of the biomarker for a subject with a CDR of 0 is typically greater than 15% to indicate a diagnosis or prognosis of AD. In some instances, the percent elevation or depression is 15%, 16%, 17%, 18%, 19%, 20%, 21%, or 22%. In other instances, the percent elevation or depression is 23%, 24%, 25%, 26%, 27%, 28%, 29% or 30%. In still other instances, the percent elevation or depression is greater than 30%. In alternative instances, the percent elevation or depression is greater than 50%.

b. Using Biomarkers to Monitor AD

Another embodiment of the invention encompasses a method for monitoring AD comprising quantifying the level of an AD biomarker in a bodily fluid of a subject and comparing the quantified level of the biomarker to a previously quantified biomarker level of the subject to determine if the quantified level is elevated or depressed in comparison to the previous level. The subject may be diagnosed with AD, or alternatively, may have no clinical signs of AD. The comparison may give an indication of disease progression. Therefore, the comparison may serve to measure the effectiveness of a chosen therapy. Alternatively, the comparison may serve to measure the rate of disease progression. For example, a depressed ATIII level, in comparison to a previous level, may indicate an abatement of disease progression. Alternatively, an elevated YKL-40 level, in comparison to a previous level, may indicate an abatement of disease progression.

In the context of monitoring AD, the percent elevation or depression of an AD biomarker compared to a previous level may be from 0% to greater than about 50%. In one embodiment, the percent elevation or depression is from about 1% to about 10%. In another embodiment, the percent elevation or depression is from about 10% to about 20%. In yet another embodiment, the percent elevation or depression is from about 20% to about 30%. In still another embodiment, the percent elevation or depression is from about 30% to about 40%. In yet still another embodiment, the percent elevation or depression is from about 40% to about 50%. In a further embodiment, the percent elevation or depression is greater than 50%.

### III. Kits for Detecting or Monitoring AD

Another aspect of the invention encompasses kits for detecting or monitoring AD in a subject. A variety of kits having different components are contemplated by the current invention. Generally speaking, the kit will include the means for quantifying one or more AD biomarkers in a subject. In another embodiment, the kit will include means for collecting a bodily fluid, means for quantifying one or more AD biomarkers in the bodily fluid, and instructions for use of the kit contents. In certain embodiments, the kit comprises a means for quantifying AD biomarker enzyme activity. Preferably, the means for quantifying biomarker enzyme activity comprises reagents necessary to detect the biomarker enzyme activity. In certain aspects, the kit comprises a means for quantifying the amount of AD biomarker protein. Preferably, the means for quantifying the amount of biomarker protein comprises reagents necessary to detect the amount of biomarker protein.

In one embodiment, the kit comprises means to quantify the level of ATIII in a bodily fluid of a subject. The level of ATIII refers to either the enzyme activity of ATIII or the protein concentration of ATIII. In another embodiment, the kit comprises a means to quantify the level of at least one AD biomarker. In yet another embodiment, the kit comprises means to quantify the level of at least two, at least three, at least four, at least five or at least six AD biomarkers. In still yet another embodiment, the kit comprises means to quantify the level of at least seven, at least eight, at least nine, or at least ten AD biomarkers. In still another embodiment, the kit comprises means to quantify the level of ten or more biomarkers. In certain embodiments, the kit comprises the means to quantify the level of one or more biomarkers from the group consisting of ATIII, ACT, ZAG, CNDP1, Aβ42, YKL-40 and tau. In each of the above embodiments, the AD biomarker level refers to either the biomarker enzyme activity or the biomarker protein concentration. The means necessary to detect either enzyme activity or protein concentration are discussed in Part II above.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate the invention.

### Example 1

### Materials and Methods for Example 1

CSF Sampling: Human CSF samples were obtained by lumbar puncture (LP) from subjects enrolled in the Memory and Aging Project at Washington University as part of an ongoing biomarker study. The study protocol was approved by the Human Studies Committee at Washington University, and written and verbal informed consent was obtained from each participant at enrollment. In 6 individuals, two samples were obtained from the same individual 2 weeks apart. Two weeks is an arbitrary time span, chosen to allow the skin, subcutaneous tissue, and meninges to have adequate time for repair prior to a second LP. Other time periods may certainly be used. All LPs were performed at the same time of the day with no fasting requirement. 25-35 ml of CSF was obtained from all participants with either a 22 or a 25 guage spinal needle. All CSF samples were free of blood contamination. After collection, CSF samples were briefly centrifuged at 1,000×g to pellet any cell debris, frozen, and stored in polypropylene tubes at −80° C. in 0.5-ml aliquots until analysis. The age of these six individuals ranged from 64 to 91 years. The cognitive state of the subjects was rated using a Clinical Dementia Rating (CDR) scale in which an individual's cognition is rated as normal (CDR 0), or demented with severities of very mild, mild, moderate or severe (CDR 0.5, 1, 2 or 3, respectively). Individuals diagnosed with possible/probable dementia of the Alzheimer's type (DAT) are usually CDR 1 or greater. Of the 6 individuals who had CSF sampling on 2 occasions, 2 weeks apart, four of these subjects had a CDR score of 0, and the other two were rated as CDR 0.5. The protein content in each CSF sample was determined with the micro-BCA protein assay kit (Pierce), and it ranged from 570 to 1,000 μg/ml.

Multiaffinity Immunodepletion of CSF Proteins: Because albumin, IgG, α1-antitrypsin, IgA, transferrin, and haptoglobin collectively account for ˜80% of the total CSF protein content, these proteins were selectively removed to enrich for proteins of lower abundance. An antibody-based multiaffinity removal system (Agilent Technologies, Palo Alto, Calif.) was used according to the manufacturer's instructions. Briefly 1.5-2 ml of CSF was concentrated and buffer-exchanged with Agilent Buffer A to a final volume of 50 μl using Amicon Ultra-4 centrifugal filter units (10-kDa cut-off) (Millipore). Samples were then diluted to 200 μl with Buffer A and passed through an Ultra-free MC microcentrifuge filter (0.22 μm) (Millipore) to remove particulates. The filtrate was injected at 0.25 ml/min onto a 4.6×50-mm multiple affinity removal column equilibrated at room temperature with Agilent Buffer A on a Microtech (Vista, Calif.) Ultra-Plus HPLC system. CSF devoid of high abundance proteins (flow-through) was collected between 1.5 and 6 min. After 9 min of elution with Buffer A, the eluant was changed to Agilent Buffer B at 1 ml/min. The six bound proteins were eluted from the column between 10 and 14 min. After 3.5 min, the column was regenerated with Buffer A.

2-D DIGE: Depleted CSF samples were buffer-exchanged and concentrated with lysis buffer (30 mM Tris-Cl, pH 7.8, 7 M urea, 2 M thiourea, 4% CHAPS containing protease inhibitors (catalog number 697498, Roche Diagnostics) and phosphatase inhibitors (catalog numbers 524624 and 524625, EMD Biosciences, Darmstadt, Germany) using Amicon Ultra-4 centrifugal filter units (10-kDa cut-off) (Millipore). The protein concentration was determined with a modified Lowry method (PlusOne 2D-Quant kit, Amersham Biosciences). Fifty micrograms of protein from each sample was labeled with 400 pmol of one of three N-hydroxysuccinimide cyanine dyes for proteins (Amersham Biosciences), diluted with rehydration buffer (7 M urea, 2 M thiourea, 4% CHAPS, 2.5% DTT, 10% isopropanol, 5% glycerol, and 2% PharmalytepH 3-10), combined according to experimental design, and equilibrated with IPG strips (24 cm; pH 3-10, nonlinear). The three samples that were equilibrated with each IPG strip consisted of two depleted CSF samples from the same individual (Cy2 and Cy5) and a pooled sample (pooled using an equal volume aliquot of each of the 12 CSF samples) (Cy3) as the internal standard. First dimension isoelectric focusing was performed at 65.6 kV-h in an Ettan IPGphor system (Amersham Biosciences). The strips were then treated with reducing and alkylating solutions prior to the second dimension (SDS-PAGE). After equilibration with a solution containing 6 M urea, 30% glycerol, 2% SDS, 50 mM Tris-Cl, pH 7.8, 32 mM DTT, the strips were treated with the same solution containing 325 mM iodoacetamide instead of DTT. The strips were overlayered onto a 10% isocratic or gradient SDS-PAGE gel (20×24 cm), immobilized to a low fluorescence glass plate and electrophoresed for 18 h at 1 watt/gel. The Cy2-, Cy3-, and Cy5-labeled images were acquired on a Typhoon 9400 scanner (Amersham Biosciences) at the excitation/emission values of 488/520, 532/580, 633/670 nm, respectively.

Image Analyses: Intragel spot detection and quantification and intergel matching and quantification were performed using Differential In-gel Analysis (DIA) and Biological Variation Analysis (BVA) modules of DeCyder software version 5.01 (Amersham Biosciences) as described previously (Alban et al., (2003) Proteomics 3, 36-44; Tonge et al., (2001) Proteomics 1, 377-396). Briefly in DIA, the Cy2, Cy3, and Cy5 images for each gel were merged, spot boundaries were automatically detected, and normalized spot volumes (protein abundance) were calculated. During spot detection, the estimated number of spots was set at 3,500, and the exclude filter was set as follows: slope, >1.1; area, <100; peak height, <100; and volume, <10,000. This analysis was used to calculate abundance differences in given proteins between two samplings from the same individual. The resulting spot maps were exported to BVA. Matching of the protein spots across six gels was performed after several rounds of extensive land marking and automatic matching. Dividing each Cy2 or Cy5 spot volume with the corresponding Cy3 (internal standard) spot volume within each gel gave a standard abundance, thereby correcting intergel variations. For each of the CSF samples, a profile was created that consisted of standard abundance for all of the matched spots.

Protein Digestion and Mass Spectrometry: Gel features were selected in the DeCyder software and the X and Y coordinates were saved in a file for spot excision. After translation using in-house software (Imagemapper), the central core (1.8 mm) of the selected gel features was excised with a ProPic robot (Genomics Solutions, Ann Arbor, Mich.) and transferred to a 96-well PCR plate. The gel pieces were then digested in situ with trypsin using a modification of a published method (Havlis et al., (2003) Anal. Chem. 75, 1300-1306). To maximize specificity, sensitivity, and sequence coverage of the digested proteins, the resulting peptide pools were analyzed by tandem MS using both MALDI and ESI. Spectra of the peptide pools were obtained on a MALDI-TOF/TOF instrument (Proteomics 4700, Applied Biosystems, Foster City, Calif.). The initial spectra were used to determine the molecular weights of the peptides (to within 20 ppm of their theoretical masses). Selected precursor ions were then focused in the instrument using a timed ion selector, and peptide fragmentation spectra were produced after high energy (1.5-keV) collision-induced dissociation. ESI-MS was performed using an advanced capillary LC-MS/MS system (Eksigent nano-LC 1D Proteomics, Eksigent Technologies, Livermore, Calif.). A nanoflow (200 nl/min) pulse-free liquid chromatograph was interfaced to a quadrupole time-of-flight mass spectrometer (Q-STAR XL, Applied Biosystems) using a PicoView system (New Objective, Woburn, Mass.). Sample injection was performed with an Endurance autosampler (Spark Holland, Plainsboro, N.J.). The peptide fragmentation spectra were processed using Data Explorer version 4.5 or Analyst software (Applied Biosystems). After centroiding and background subtraction, the peak lists were used to search databases with MASCOT version 1.9 (Matrix Sciences, Boston, Mass.). Peptide sequences were qualified by manual interpretation of raw non-centroided spectra.

CSF Biomarker Assessment (ELISA): CSF samples were analyzed for total tau, amyloid β42 (Aβ42), α1-chymotrypsin (ACT), antithrombin III (ATM), and gelsolin by commercial enzyme-linked immunosorbant assay (ELISA) (Innotest, Innogenetics, Ghent, Belgium). For all biomarker measures, samples were continuously kept on ice, and assays were performed on sample aliquots after a single thaw from initial freezing.

Threshold Selection: The DIA software performs a log transformation of the volume ratios and uses them to generate a frequency histogram. A normal distribution is fitted to the main peak of the frequency histogram. After normalization, this fitted distribution curve centers on 0, which represents proteins with unaltered abundance. Model standard deviation (S.D.) is then derived based on the normalized model curve. 2 S.D., the volume ratio for 2 S.D. based on the raw data, is the software-recommended cut-off. In a normally distributed data set, 95% of data points would fall within this value. Based on the observation that 2 S.D. ranged from 1.31 to 1.52 for the six individuals who were compared 2 weeks apart, gel features changing by >1.5 in spot volume were considered significant.

p Value Determination for Intraindividual Variation: The statistical significance of observing different levels of the same protein in multiple intraindividual comparisons was estimated by describing the data as a binomial distribution and calculating the probability of the observed events. Our null hypotheses are as follows: 1) all intra-individual comparison experiments are independent from each other, and 2) in any intra-individual comparison, protein levels should not change; therefore any observed change should be random and represent system fluctuation rather than a property of an individual protein. For any given experiment (intra-individual comparison) that follows the null hypotheses, the probability of any protein changing its expression level is p_{c}. This value can be estimated by maximum a posteriori estimation; i.e. based on the observed number of protein spots detected in a given gel and the observed number of spots determined to have altered abundance (i.e. having a >1.5 spot volume ratio) between the two time points in an intraindividual comparison, we calculated p_{c}. such that the probability of observing the experimental data given p_{c}. is maximized. In N independent trials (in this case six intraindividual comparisons), the probability of observing the same protein having changed abundance in n or more individuals is as follows.

$$p ( i \geq n / N ) = {\sum }_{i = n … N} ( \begin{matrix}N \\ n\end{matrix} ) {{p}_{c}^{n} ( 1 - {p}_{c} )}^{N - n} \qquad ( Eq . 1 )$$

p_{c}. is determined to be 0.0140, and N is 6. Because not all of the “changed” spots are identified by MS/MS, this p value is likely an underestimation of the significance.

Hierarchical Clustering and Multidimensional Scaling Analysis: Hierarchical clustering was performed using Spotfire (Spotfire, Somerville, Mass.) software. Unweighted pair group method with arithmetic mean (UPGMA) was selected as the clustering method, and Euclidean distance was selected as the similarity measure. BRB Arraytools (linus.nci.nih.gov/BRB-ArrayTools.html) were used for multidimensional scaling analysis. Euclidean distance was used to measure similarity.

### Results:

Prior to comparing CSF samples between individuals to identify patterns of disease-associated proteins, it is important to examine variation within individuals over a short period of time so that one can better interpret potential changes in CSF between individuals, as well as changes within a given individual over a longer time span. In a first study, we analyzed 12 CSF samples, composed of pairs of samples from six individuals, obtained 2 weeks apart. Multiaffinity depletion, two-dimensional DIGE, and tandem mass spectrometry were used. A number of proteins whose abundance varied between the two time points were identified for each individual. Some of these proteins were commonly identified in multiple individuals. More importantly, despite the intraindividual variations, hierarchical clustering and multidimensional scaling analysis of the proteomic profiles revealed that two CSF samples from the same individual cluster the closest together and that the between-subject variability is much larger than the within-subject variability. Among the six subjects, comparison between the four cognitively normal and the two very mildly demented subjects also yielded some proteins that have been identified in previous AD biomarker studies.

One objective of this study was to evaluate variability in the CSF proteome associated with longitudinal collection of CSF from individuals. Utilizing single gel, multiple image analysis, we were able to identify within-subject differences with a high degree of confidence. By including a pooled sample in every gel as an internal standard, we were able to match and perform relative quantification of spots across gels and compare the degree of within-subject variation with that of between-subject variation. This study is an important component in the long term research program of the applicants to identify biomarkers for preclinical and very mild AD.

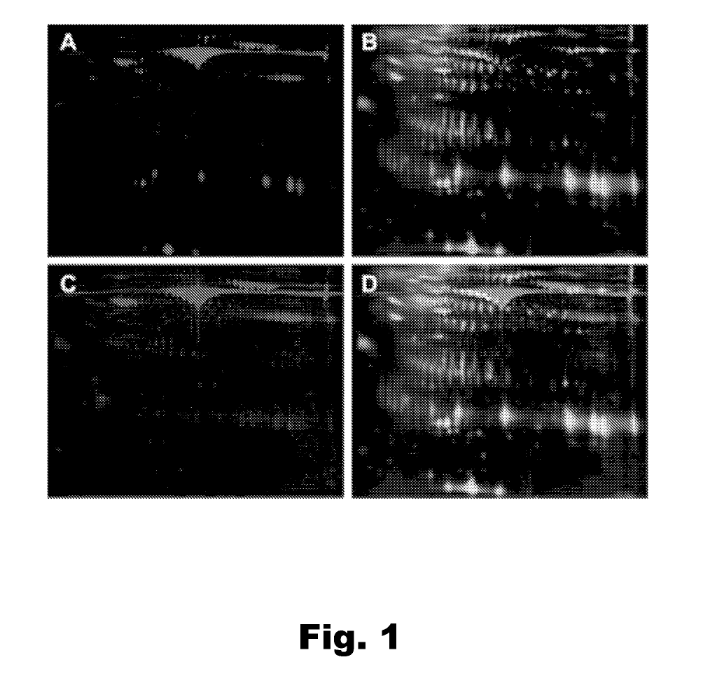

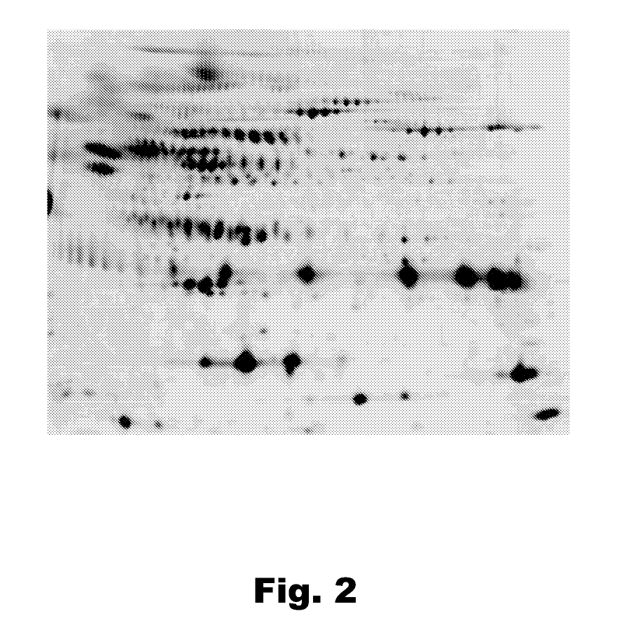

Because high abundance proteins comprise a large portion of the protein content of CSF and thus dominate 2-D gel images, we removed these proteins to increase our ability to detect and quantify proteins of lower abundance. We used a column-based multiple immunoaffinity system to remove six proteins (albumin, IgG, α₁-antitrypsin, IgA, transferrin, and haptoglobin) from human CSF. This depletion technique has been shown in a proteomic study on serum to be superior to three other similar depletion methods and resulted in a 76% increase in the number of protein spots detected (Chromy et al., (2004) J. Proteome Res. 3, 1120-1127). When loading the same amount of total protein, our CSF study showed a 99% increase in the number of spots detected (FIG. 1): up to ˜2,100 spots were detected on a gel. FIG. 2 is a representative 2-D DIGE image of a postdepletion CSF sample used in this study.

Two CSF samples were taken from the same individual 2 weeks apart (time point 1 (T1) and time point 2 (T2)) and were analyzed on the same gel together with a pooled sample as an internal standard. Six individuals were analyzed, translating into 12 CSF samples and six gels. The internal standard was a pool that included an equal volume aliquot of all 12 CSF samples. Pairwise comparisons of individual spots were made between T1 and T2 samplings for each individual to identify differences in protein abundance. A number of spots were selected for MS/MS analysis for each individual based on the following two criteria: 1) the -fold change of the spot volume between the two time points was greater than 1.5 (see “Experimental Procedures” for threshold selection), and 2) the protein spot was well resolved and appeared as a symmetrical peak. A total of 104 spots met these criteria, ranging from eight to 34 spots per gel, and 73 of them were identified by MS/MS. Table I presents some of the identified proteins, along with the number of individuals sharing a greater than 1.5-fold change. The -fold change is the change in protein abundance at T2 compared to T1 (the direction of change was ignored).

TABLE 1
Proteins identified by MS/MS that vary in abundance within individuals
over a 2-week time period.

| Protein | GenBank ™ ID | Number | -Fold change | p value |
|---|---|---|---|---|
| Transthyretin | 339685 | 6 | 1.92 ± 0.46 | 7.53 × 10⁻¹² |
| Chromogranin A | 180529 | 3 | 1.79 ± 0.09 | 5.32 × 10⁻⁵ |
| Complement component C4A | 443671 | 3 | 1.73 ± 0.31 | 5.32 × 10⁻⁵ |
| Hemopexin precursor | 386789 | 3 | 1.63 ± 0.09 | 5.32 × 10⁻⁵ |
| Osteopontin-a | 992948 | 3 | 1.50 ± 0.11 | 5.32 × 10⁻⁵ |
| β fibrinogen precursor | 182430 | 2 | 3.33 ± 1.21 | 0.00283 |
| Apolipoprotein E | 178849 | 2 | 3.13 ± 0.03 | 0.00283 |
| Complement component 3 precursor | 4557385 | 2 | 2.81 ± 1.60 | 0.00283 |
| Semaphorin L | 3551779 | 2 | 2.06 ± 0.74 | 0.00283 |
| Aprotinin | 58005 | 2 | 2.05 ± 0.51 | 0.00283 |
| Prostaglandin D₂ synthase | 54696706 | 2 | 1.97 ± 0.17 | 0.00283 |
| a₂-Macroglobulin | 177872 | 2 | 1.75 ± 0.18 | 0.00283 |
| Fibulin 1 precursor | 106018 | 2 | 1.64 ± 0.31 | 0.00283 |
| Chromogranin B precursor | 4502807 | 2 | 1.64 ± 0.12 | 0.00283 |
| Ubiquitin | 13569612 | 2 | 1.60 ± 0.06 | 0.00283 |
| Fibrinogen γ | 71828 | 1^{a} | 3.87 ± 2.55 | 0.0811 |
| Apolipoprotein H precursor | 4557327 | 1^{a} | 2.55 ± 0.30 | 0.0811 |
| PRO 1400 | 6650772 | 1^{a} | 1.99 ± 0.58 | 0.0811 |
| Transferrin | 31415705 | 1^{a} | 1.66 ± 0.18 | 0.0811 |
| Pyruvate kinase | 2117873 | 1 | 6.02 | 0.0811 |
| Complement C8-β propeptide | 29575 | 1 | 5.49 | 0.0811 |
| Angiotensinogen | 532198 | 1 | 4.72 | 0.0811 |
| Coagulation factor XII | 180359 | 1 | 2.15 | 0.0811 |
| Scrapie-responsive protein 1 | 37183198 | 1 | 1.62 | 0.0811 |
| Cerebroside sulfate activator protein | 337760 | 1 | 1.60 | 0.0811 |
| α albumin | 4501987 | 1 | 1.52 | 0.0811 |

^{a}Several isoforms were identified in the same individual

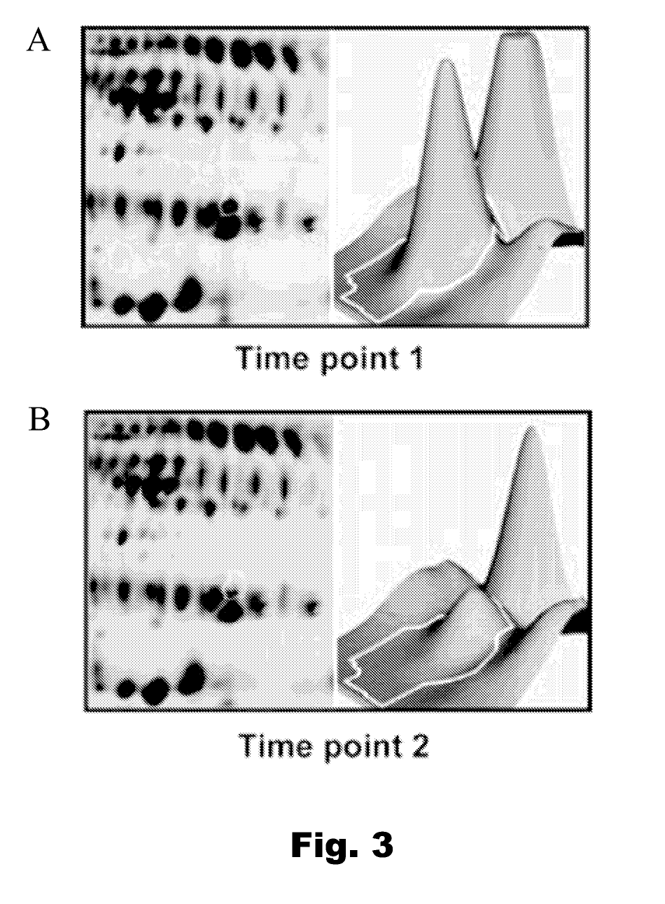

There was a certain degree of variability in the CSF proteome detected within an individual over a 2-week period. Representative gel images and 3-D representations of one of such proteins are shown in FIG. 3. There was a 3.1-fold change of the levels of this ApoE spot between the two time points. Although many protein constituents in the CSF are derived from plasma, some of these changed proteins (Table 1) have been shown to be enriched in CSF or derived from nervous tissue, such as prostaglandin D₂ synthase, transthyretin, apolipoprotein E (apoE), chromogranin A, chromogranin B, semaphorin L, and scrapie-responsive protein 1. Interestingly a number of the same proteins are found to vary in multiple individuals. We calculated the p value of a given protein appearing as changed by chance in one or more individuals (Table 1) and found that it is statistically significant when a protein is shown as changed in two or more individuals (using a cut-off p value of 0.05). The protein found to vary most often is transthyretin (six of six individuals). Other commonly variable (isoforms of) proteins include chromogranins A and B, complement component 3 and C4A, hemopexin precursor, osteopontin a, β fibrinogen precursor, apoE, semaphorin L, prostaglandin D₂ synthase, α₂-macroglobulin, fibulin precursor, and ubiquitin. Aprotinin, a synthetic peptide present in the lysis buffer, appeared as a changed protein. Transthyretin and apoE have been shown to promote the solubility, transport, and clearance of amyloid-β (Aβ), a molecule important in the pathogenesis of AD. For transthyretin, multiple isoforms were found to vary within individuals (in the same direction for a given individual), whereas only one isoform of apoE was found to vary. In fact, our apoE ELISA data showed that total apoE level does not vary significantly within individuals. The direction of the intra-individual changes (increase versus decrease when comparing the initial and subsequent CSF sampling) did not show any trends among individuals. These findings strongly suggest that intra-individual variations in the CSF proteome reflect the dynamic steady states of those proteins. These data also suggest that the levels of some (isoforms of) proteins in CSF tend to fluctuate more significantly than others due to the nature of their metabolism as opposed to standard errors in experiments. Such intra-individual protein abundance variations are a caveat when considering these proteins as potential disease-related biomarkers.

An important goal of this study was to quantify the degree of intra-individual variation in relation to variation between individuals. For that purpose, protein spots were matched across all six gels so that a proteomic profile containing matched spots can be created for each sample, allowing for statistical comparisons to be made between individual profiles.

One advantage of 2-D DIGE is the ability to perform intergel matching and comparison through the inclusion of an internal standard on each gel. Nevertheless the assumption in using 2-D gel image analysis to measure relative protein concentration is that matched spots (spots that are located at the same position in different gels) correspond to the identical protein. This assumption was tested by first matching all six gels using one of the pooled samples (internal standard) as a master image, from which 306 protein spots across all six gels were matched. Sixteen matched spots that were well resolved and distributed across the entire gel were selected and analyzed with MS/MS. For 14 spots, protein identifications were obtained from more than two gels. For 11 of the 14 spots, the identified proteins were the same. Examination of the 3-D gel images revealed that, for the three spots that gave different proteins there was evidence (e.g. a ridge) of another protein underneath the most prominent gel feature or matching/picking aberrations. Our conclusion is that matched, well-resolved gel features correspond to the same protein.

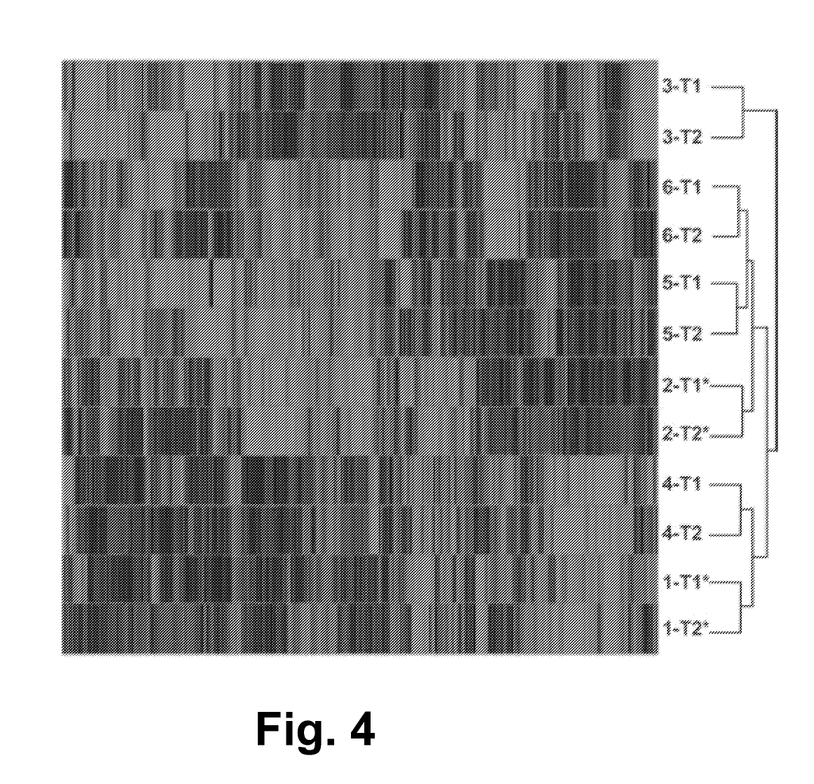

After intergel matching, a proteomic profile that consisted of standard abundance (i.e.—fold change in protein abundance compared with the pooled internal standard) was generated for matched spots for each of the 12 CSF samples. Because standard abundance is derived using the spot volume of the pooled sample as a denominator, it can be considered as the relative abundance of a protein spot. In this dataset, two statistical analyses were applied to assess the relationship among the CSF samples. First, hierarchical clustering analysis was performed on the proteomic profiles. Hierarchical clustering orders objects in a treelike structure based on similarity (i.e. in this case, “pattern similarity”). Clustering analysis is used extensively in the mining of gene expression data generated by functional genomic studies, but its application to protein expression data remains limited. Through the inclusion of an internal standard, a dataset was created that was very similar to datasets derived from GeneChip or microarray experiments and therefore allowed for the application of clustering algorithms to 2-D gel image analysis. The results, including a dendrogram and a heat map, are presented in FIG. 4. The dendrogram reveals that each pair of intraindividual CSF samples (T1 and T2) was clustered the closest together. As can be visualized in the heat map, the proteomic profiles of intra-individual samples are most similar to each other and distinctively different from other individuals' profiles. The two CDR subjects with very mild AD (i.e. CDR 0.5, marked with an asterisk) did not cluster the closest together, indicating that there were no obvious global changes of protein expression between the CDR 0.5 and CDR 0 samples in this initial study. To rule out the possibility that the two samplings from the same subject cluster together simply because they were run on the same gel, we analyzed T1 samples from two subjects on one gel and T2 samplings from these two subjects on another gel. Clustering analysis of the proteomic profiles showed that longitudinal samples from the same subject still cluster the closest together. This further demonstrates the validity of the intergel comparison.

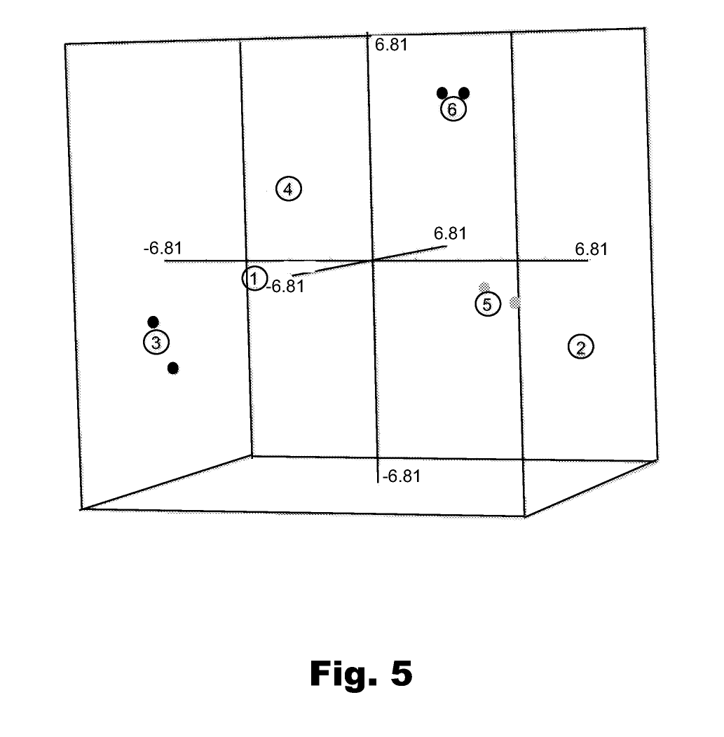

Second, multidimensional scaling analysis was performed on the proteomic profiles. Like hierarchical clustering, multidimensional scaling is commonly used in microarray research as an analysis tool. Its purpose is to capture as much variation in the data as possible in a minimal number of dimensions (two or three) so that trends in data are more obvious. In effect, one is attempting to reduce the dimensionality of the data to summarize the most influential (i.e. defining) components while simultaneously filtering out noise. The distance between two samples can be thought to represent the similarity between them; the smaller the distance, the more similar the samples. FIG. 5 is a 2-D projection of the 3-D scatter plot of the proteomes of the 12 CSF samples. CSF samples from the same subject are represented with the same color. As can be observed from the graph, there is a short distance between two samples from the same individual, and this distance varies among individuals. However, the distance between intraindividual CSF samples is much smaller than between samples from different individuals. These data highlight that intraindividual variation in the CSF proteome is much smaller than the interindividual variation.

Because our sample set included four CDR 0 subjects (eight CSF samples) and two CDR 0.5 subjects (four CSF samples), we were able to evaluate possible CDR group-associated differences. As mentioned earlier, 306 spots were matched across six gels/12 individual CSF samples. For each spot in a given CSF sample, there is a corresponding standard abundance (representing the relative abundance of this protein spot) that can be compared across the 12 samples. Therefore, we were able to perform at test (in the BVA module of the DeCyder software) on the matched spots by designating the eight CDR 0 CSF samples as group 1 and the four CDR 0.5 samples as group 2. We selected the top ranking (p<0.02) and well resolved spots and subjected them to MS/MS analysis. Eleven of the 13 spots selected were found to represent eight different proteins (Table 2). Interestingly four of these proteins have been shown to have altered levels in AD CSF, including α₁β-glycoprotein, prostaglandin D₂ synthase, cystatin C, and β₂-microglobulin. Consistent with previous studies, α₁β-glycoprotein is decreased in the CDR 0.5 group, whereas cystatin C and β₂-microglobulin are increased. Prostaglandin D₂ synthase was found to increase in the CDR 0.5 group, which is consistent with a previous study, whereas another study reported a decrease of prostaglandin D₂ synthase. Several isoforms of chitinase 3-like 1, also known as GP-39 cartilage protein, were found to be increased in the CDR 0.5 group. This protein is primarily produced by human chondrocytes and synovial fibroblasts and has been shown to be a target antigen in patients with rheumatoid arthritis.

TABLE 2
CSF proteins found to be differentially expressed between
CDR 0.5 and CDR 0 groups.

| Spot | | GenBank ™ | | Direction |
|---|---|---|---|---|
| ID | Protein | ID | p value | of change |
| 1 | α₁β-Glycoprotein | 69990 | 0.011 | Decrease |
| | Complement component | 4557385 | 0.011 | Decrease |
| | 3 precursor | | | |
| 2 | Apolipoprotein H precursor | 4557327 | 0.0071 | Decrease |
| 3 | Chitinase 3-like 1 (YKL-40) | 4557018 | 0.011 | Increase |
| 4 | Chitinase 3-like 1 (YKL-40) | 4557018 | 0.014 | Increase |
| 5 | Prostaglandin D₂ synthase | 55962672 | 0.015 | Increase |
| | Chitinase 3-like 1 (YKL-40) | 4557018 | 0.015 | Increase |
| 6 | Prostaglandin D₂ synthase | 54696706 | 0.00022 | Increase |
| 7 | Prostaglandin D₂ synthase | 54696706 | 0.016 | Increase |
| 8 | Prostaglandin D₂ synthase | 54696706 | 0.011 | Increase |
| 9 | Cystatin C | 181387 | 0.019 | Increase |
| 10 | Thioredoxin | 231098 | 0.0058 | Increase |
| 11 | β₂-Microglobulin | 34616 | 0.0022 | Increase |

Although this sample size was very small, five of eight differentially expressed proteins identified by MS/MS have been implicated in previous AD biomarker studies, and for three of them, these results (increase versus decrease) are consistent with previous reports. Importantly only one of these proteins (i.e. prostaglandin D₂ synthase) was identified to display intra-individual variation. These preliminary differences that were found need to be validated with a much larger sample set; however, these results suggest that, given appropriate sample selection, protein quantification, and profile comparison, one may not require a large set of samples to identify disease-related biomarkers.

### Example 2

### Materials and Methods for Example 2

Subjects: Research subjects were participants at the Alzheimer's Disease Research Center (ADRC) at the Washington University School of Medicine (WUSM) and were recruited by the ADRC to this study. All subjects gave informed consent to participate in this study and all protocols were approved by the institutional review board for human studies at Washington University. Study investigators were blind to the cognitive status of the participants, which was determined by ADRC clinicians in accordance with standard protocols and criteria, as described previously (Berg et al., Arch Neurol (1998) 55:326-335; Morris et al., Ann Neurol (1988) 24:17-22). Subjects were assessed on clinical grounds to be cognitively normal in accordance with a Clinical Dementia Rating (CDR) of 0 (n=55) or to have very mild (CDR 0.5; n=20) or mild (CDR 1; n=19) dementia that is believed to be caused by AD as described (Morris et al., Arch Neurol (2001) 58:397-405).

CSF and plasma collection: CSF (20-35 ml) was collected at 8:00 AM after overnight fasting, as described previously (Fagan et al., Ann Neurol (2000) 48:201-210). Lumbar punctures (LPs) (L4/L5) were performed by a trained neurologist using a 22-gauge Sprotte spinal needle. CSF samples were free from any blood contamination. Samples were gently inverted to avoid gradient effects, briefly centrifuged at low speed to pellet any cellular elements, and aliquoted (500 μl) into polypropylene tubes before freezing at −80° C. Fasted blood (10-15 ml) was also obtained from each subject just before LP, and plasma was prepared by standard centrifugation techniques. Plasma samples were aliquoted (500 μl) into polypropylene tubes before freezing at −80° C.

Multi-affinity immunodepletion of abundant CSF proteins: Since albumin, IgG, α1-antitrypsin, IgA, transferrin and haptoglobin collectively account for ˜80% total CSF protein content, they were selectively removed in order to enrich for proteins of lower abundance. An antibody-based multi-affinity removal system (Agilent Technologies, Palo Alto, Calif.) was employed according to the manufacturer's instructions, as described in the materials and methods for examples 1-3.

2D-DIGE: Twelve CSF samples were analyzed by 2D-DIGE: CDR 1 subjects (n=6) and age-matched CDR 0 controls (n=6). These samples were selected based on Aβ42 concentration, i.e. CDR 1 samples were selected whose Aβ42 concentration is less than 500 pg/ml and the converse for CDR 0 samples. Fifty μg of protein from each depleted CSF sample was labeled with 400 pmol of one of three N-hydroxsuccinimide cyanine dyes for proteins (GE Healthcare, Piscataway, N.J.), mixed with 30 μg of unlabled protein from the same sample, diluted with rehydration buffer, combined according to experimental design, and equilibrated with immobilized pH gradient (IPG) strips (24 cm; pH 3-10, nonlinear). The three samples that were equilibrated with each IPG strip consisted of a CDR 0 sample, a CDR 1 sample and a pooled sample (pooled using an equal volume aliquot of each of the 12 CSF samples) (labeled with Cy3) as the internal standard. To avoid possible dye-related bias, half of the CDR 0 samples were labeled with Cy2 and half with Cy5 and the same protocol was used to label the CDR 1 samples. First-dimension isoelectric focusing was performed at 65.6 kV-h in an Ettan IPGphor system (GE Healthcare). The second dimension was performed on a gradient SDS-PAGE gel (10-20%). The Cy2, Cy3, Cy5-labeled images were acquired on a Typhoon 9400 scanner (GE Healthcare).

Image analysis: Intra-gel spot detection, quantification and inter-gel matching and quantification were performed using Differential In-gel Analysis (DIA) and Biological Variation Analysis (BVA) modules of DeCyder software v5.01 (GE Healthcare), respectively as described previously (Hu et al., Mol Cell Propteomics (2005) 4:2000-9, hereby incorporated by reference in its entirety). A subset of protein spots (n=514) were matched across all six gels. Student's t-test was performed to compare the average (relative) abundance of a given spot between the two groups (CDR 1 vs. CDR 0). Spots that had a p value below 0.05 and were well resolved were selected for subsequent mass spectrometric analysis.

Protein digestion and mass spectrometry: Gel spots of interest were flagged with the DeCyder software and the X and Y coordinates were used by a robotic spot picker (ProPic; Genomics Solutions, Ann Arbor, Mich.) to cut and transfer gel features into a 96 well plate for in situ gel digestion with trypsin using a modification of a previously described method (Havlis et al. Anal Chen (2003) 75:1300-6). Briefly, excised gel cylinders (2 mm in diameter) were robotically transferred to 96 well plates (Axygen Scientific, Union City, Calif.) and over-layered with 100 μL of water. After washing and trypsin digestion (Sigma, St. Louis, Mo.) in 5 μL of 5 mM tri-ethyl-ammonium bicarbonate buffer, (pH 8.0; Sigma), an aliquot (0.5 μL) was removed from each well and placed in a microfuge tube (200 μL) containing 0.5 μL of MALDI Matrix (Agilent Technologies). The tubes were vortexed, spun in a microfuge for 30 s and spotted (1 μL) onto a stainless steel target (192 spot plate) for MALDI-TOF/TOF mass spectrometry as previously described (Bredemeyer et al., PNAS (2004) 101:11785-90). To the remaining gel samples, 12 μL of aqueous acetonitrile/formic acid (1%/1%) (Sigma) was added and the plate was incubated at 37° C. for 1 h. The samples (˜10 μL) were then transferred to polypropylene auto sampler vials, spun at 10,000×g for 15 min in a Sorvall centrifuge equipped with a HB-6 rotor, and stored at −80° C. for analysis by LC-MS/MS. LC-MS/MS was performed using a capillary LC (Eksigent, Livermore, Calif.) interfaced to a nano-LC-linear quadrupole ion trap Fourier transform ion cyclotron resonance mass spectrometer (nano-LC-FTMS) (King et al., Anal Chem (2006) 78:2171-81).

Database searching was performed using MASCOT 1.9.05 software (Matrix Science, Oxford, UK) against the NCBI non-redundant database (Mar. 24, 2005). Protein identifications were supported by at least two peptides with Mascot scores of >40 and accurate measurements of the peptide parent masses within 5 ppm. Protein identifications were confirmed by manual interpretation of the fragmentation spectra with a minimal acceptance criterion of four contiguous b or y ions for each peptide sequence.

ELISA analyses: Aβ42 and total tau concentrations in the CSF samples were analyzed by a commercially available enzyme-linked immunosorbent assay (Innotest; Innogenetics, Ghent, Belgium). Albumin ELISA was performed with an ELISA kit (Bethyl Laboratories, Montgomery, Tex.). A sandwich ELISA was developed for ACT measurement: rabbit anti-human ACT antibody (1:1000; DAKO, Carpinteria, Calif.) was used for capture and a sheep anti-human ACT antibody (1:5000; The binding site, San Diego, Calif.) was used for detection. ACT purified from human plasma was used as standard (Sigma). For ATIII measurement, a sandwich ELISA was developed: rabbit anti-human ATIII antibody (1:1000; DAKO) was used for capture and a mouse monoclonal anti-human ATIII (clone HYB 230-04, 1:5000; Assay designs, Ann Arbor, Mich.) was used for detection. ATIII purified from human plasma was used as standard (Sigma). For ZAG, a sandwich ELISA was developed: rabbit anti-human ZAG antibody (1:1000; gift from Dr. Iwao Ohkubo, Shiga University of Medical Science, Japan) was used for capture and a mouse monoclonal anti-human ZAG antibody (clone 1D4, 1:100; Santa Cruz Biotechnology, Santa Cruz, Calif.) was used for detection. ZAG purified from human seminal plasma was used as standard (gift from Dr. Iwao Ohkubo). For gelsolin ELISA, CSF samples were directly coated on the plate, followed by incubation with a mouse monoclonal anti-gelsolin antibody (clone GS-2C4, 1:2000; Sigma). Gelsolin purified from human plasma was used as standard (Sigma). For AGT measurement, a sandwich ELISA was used: mouse monoclonal anti-AGT antibody (clone F8A2, 1:285; gift from Dr. Claus Oxvig, University of Aarhus, Denmark) was used for capture and a chicken antibody (1:1200; gift from Dr. Claus Oxvig) was used for detection. AGT purified from human plasma was used as standard (Calbiochem, San Diego, Calif.). For CNDP1 ELISA, CSF samples were directly coated on the plate, followed by incubation with goat anti-human CNDPI antibody (1:500; R&D systems, Minneapolis, Minn.).

For all ELISA measurements: 1) Biotinylated secondary antibody (1:5000; Jackson ImmunoResearch, West grove, PA) and poly-HRP streptavidin (1:2000; Research Diagnostics, Concord, Mass.) were used; for color development, TMB super slow or TMB super sensitive (Sigma) were used; 2)

Samples were kept continuously on ice, and assays were performed on sample aliquots after a second thaw after initial freezing; 3) The levels of a given protein in all CSF samples were measured in the same experiment; 4) Data from a single experiment is presented and similar data have been obtained from at least one replicate experiment.

Statistical analyses: The Shapiro-Wilk test was used to test the hypothesis of a normal distribution for levels of ACT, ATIII, ZAG, CNDP1, Aβ42, and tau. The distributions were log transformed to approximate a normal distribution. Values were then adjusted for age at lumbar puncture, gender, and the number of APOE4 alleles. The residuals were tested for association with case-control status using a t-test.

The area under the receiver operating characteristic curve (AUC) for each trait was calculated using the ROCR package in R (http://www.R-project.org; (Sing et al, Bioinformatics (2005) 21:3940-41)). A method proposed by Xiong et al (Med Decis Making (2004) 24:659-69) was implemented to determine the optimum linear combination of these traits and calculate confidence intervals on the AUC. This method requires a complete dataset; removal of individuals with missing phenotype data resulted in a slight decrease in the sample size (N=83).

Immunohistochemistry: ACT immunostaining was performed on ethanol fixed paraffin sections (5 μm thickness) of human brain (frontal cortex) with a rabbit anti-human ACT antibody (1:100; Accurate chemical, Westbury, N.Y.). ATIII immunostaining was performed on formalin fixed frozen sections (12 μm thickness) of human brain (frontal cortex) using a rabbit anti-human ATIII antibody (1:500; Dako). The staining was developed chromogenically (DAB). Standard immunohistochemical protocol was employed for the above immunostaining (Han et al., Neurobiol Dis (200) 7:38-53). Thioflavine-S and X-34 staining was performed as described (Bales et al., Nature Genetics (1997) 17:263).

### Results:

Sample selection: The CSF proteomes of 6 mild AD subjects were compared to those of 6 age-matched cognitively normal subjects. The mild AD subjects were clinically characterized with a CDR score of 1, indicating mild dementia and the controls all had a CDR score of 0, indicating non-demented. To maximize the likelihood of selecting CDR 1 subjects who truly had AD pathology from CDR 0 subjects who did not, selection was based on CSF Aβ42 concentrations. A recent study comparing in vivo amyloid imaging with CSF levels of various biomarkers showed that Aβ42 is an excellent marker of cerebral amyloid deposition, independent of clinical diagnosis Fagan et al., Ann Neurol (2006) 59:512-19). The CSF samples were analyzed individually (as opposed to pooling the samples) to increase the statistical power of the analysis.

Candidate biomarkers identified. Upon 2D-DIGE analysis, 514 protein spots were matched across all six gels. Since a pooled sample was included on each gel, the relative abundance of the matched spots were normalized in all 12 individual CSF samples. Student's t-test (using p<0.05 to define statistical significance) was performed to examine whether the average (relative) abundance of a given protein spot in the two CDR groups was significantly different. Twenty-one spots were found that displayed differential abundance (p values <0.05) between the two groups. Three spots were excluded due to poor gel resolution and the remaining 18 were selected for excision, in gel tryptic digestion, and mass spectrometric identification.

To estimate whether most of these proteins are truly different in abundance as opposed to artifacts, the false discovery rate (FDR) was assessed in this study by using data from our previous study with this method (see examples 1-3) to approximate a “same-same comparison”. In that study, the same comparative proteomic approach (depletion—followed by 2D-DIGE—MS/MS) was applied to the analysis of 12 CSF samples, composed of pairs of samples from the same six individuals, obtained 2 weeks apart. When the 6 CSF samples (as a group) obtained at time point 1 were compared to the 6 samples (as a group) obtained at time point 2, it is similar to a ‘same-same comparison’ because CSF samples from the same subject should be quite similar. When that comparison was performed, 0 spots were found (out of 306 matched spots) that were differentially expressed between the two groups (using a p value of 0.05 as cutoff). This result indicates that the false positive rate in the study (when using a p value of 0.05 as cut-off) is likely to be very low.

Protein identifications of the 18 spots are shown in Table 3. The protein identity of 16 of the 18 spots were successfully determined. For some spots, multiple proteins have been identified within one spot, most likely due to comigration of certain proteins. In addition, some proteins were identified in multiple proteins spots, including α1-antichymotrypsin (ACT) and antithrombin III (ATM), likely reflecting the existence of different protein isoforms (e.g., post-translational modifications). A total of 11 proteins were identified as candidate biomarkers for mild AD. Levels of gelsolin (GSN) and carnosinase I (CNDP1) were found to be lower in the mild AD group compared to the CDR 0 group whereas levels of the other candidate markers were higher in the CDR 1 group compared to controls. The relative difference in abundance between the averages of the two groups observed in 2D-DIGE is moderate, most within ±2 fold.

TABLE 3
List of proteins that were identified through 2D-DIGE and mass
spectrometry analysis to have differential abundance in mild AD
and control CSF samples.

| | | Change in AD in | |
|---|---|---|---|
| | | the CDR 1 group | |
| | | compared to the | P value |
| Spot | Protein | CDR 0 group | (student's t-test) |
| 1 | Antithrombin III | increased | 0.028 |
| 2 | Antithrombin III | increased | 0.023 |
| 3 | Antithrombin III | increased | 0.033 |
| | Angiotensinogen | | |
| 4 | α-1-antichymotrypsin | increased | 0.031 |
| 5 | Carnosinase I | decreased | 0.018 |
| | Secretogranin III | | |
| | Kininogen | | |
| 6 | α-1-antichymotrypsin | increased | 0.031 |
| 7 | Antithrombin III | increased | 0.022 |
| 8 | α-1-antichymotrypsin | increased | 0.0091 |
| 9 | No ID | increased | 0.0041 |
| 10 | α-1-antichymotrypsin | increased | 0.015 |
| 11 | α-1-antichymotrypsin | increased | 0.011 |
| | Secretogranin III | | |
| 12 | α-1-antichymotrypsin | increased | 0.0013 |
| | Secretogranin III | | |
| | Kininogen | | |
| 13 | Angiotensinogen | increased | 0.005 |
| | Kinongen | | |
| 14 | Zinc α2-glycoprotein | increased | 0.027 |
| 15 | Zinc α2-glycoprotein | increased | 0.014 |
| | Chromagranin B | | |
| 16 | Gelsolin | decreased | 0.028 |
| 17 | No ID | decreased | 0.048 |
| 18 | Neuronal pentraxin | decreased | 0.043 |
| | Prostaglandin D2 | | |
| | synthase | | |
| | β trace | | |
| | Secretogranin III | | |

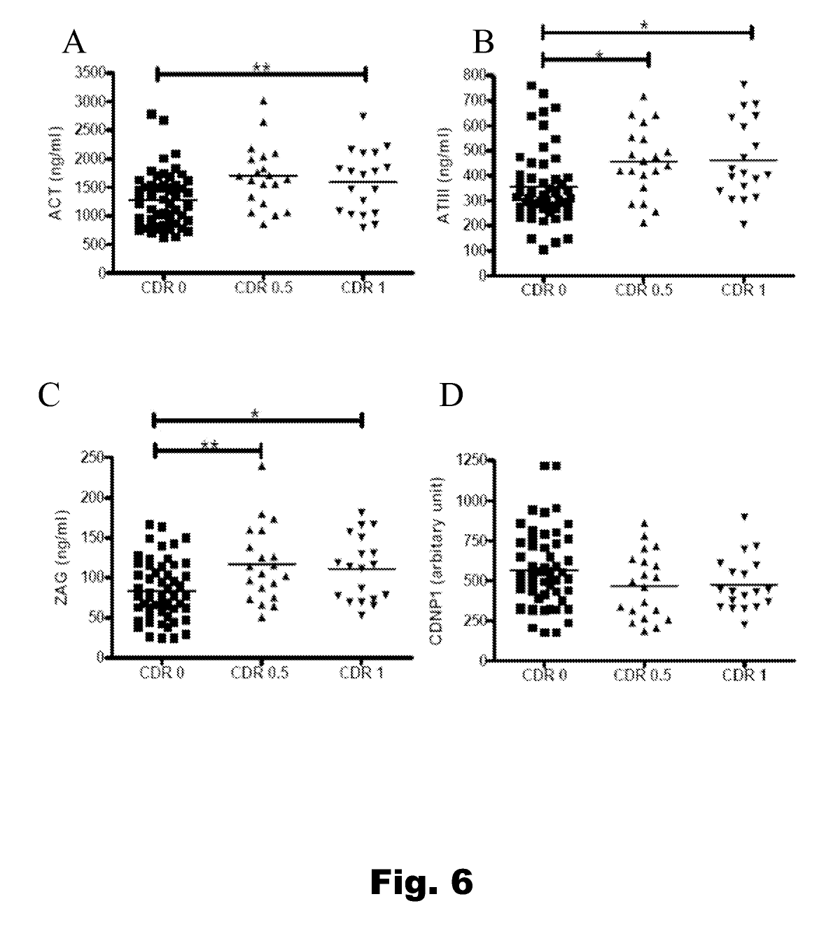

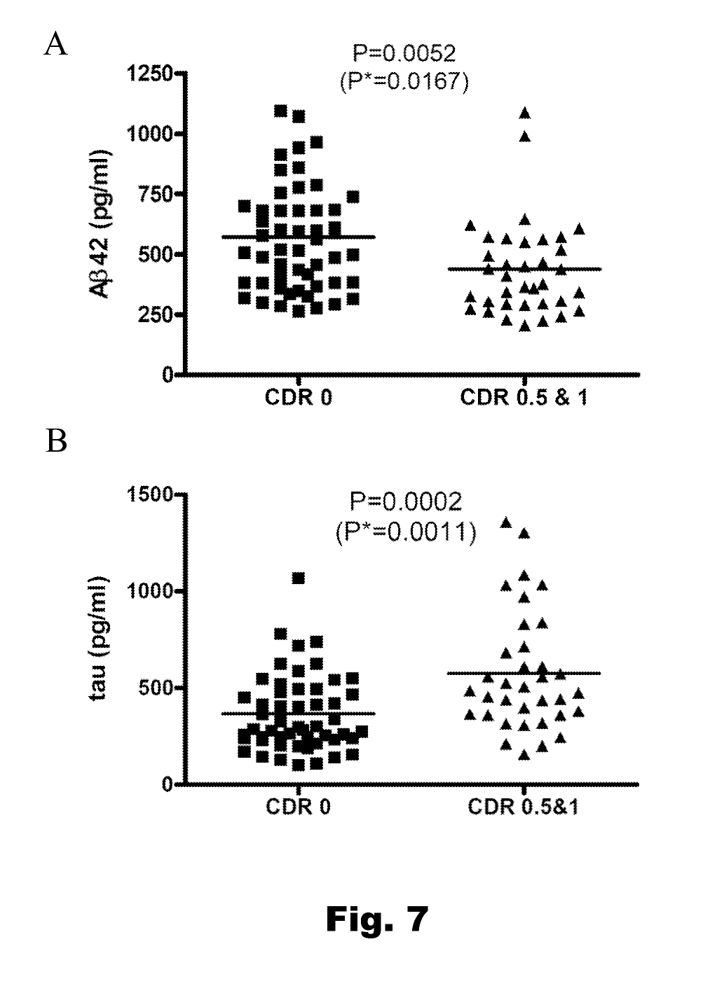

Validation of candidate biomarkers with ELISA. In order to confirm the findings with another method and also to be more quantitative, the levels of candidate biomarkers were first assayed in the original 12 CSF samples (CDR 0 (n=6), CDR1 (n=6)) using ELISA. Based on the availability of antibodies, the following candidates were assessed: ACT, ATIII, Zinc-α2-glycoprotein (ZAG), CNDP1, GSN and angiotensinogen (AGT). Then, a larger test set of CSF samples of cognitively normal subjects (CDR 0, n=49) were assayed versus subjects with very mild (CDR 0.5, n=19) or mild dementia (CDR 1, n=13) judged to be due to AD. The goal was to determine whether the findings could be validated in a larger, independent sample set. This level of validation is very important and has not been done before in previous AD proteomic biomarker studies to our knowledge. For all the candidate biomarkers evaluated, when the larger sample set was assayed, the same trend was observed as when the 12 original samples were assayed. Therefore, values from the 12 original samples were combined with the test sample set in the subsequent graphs. In addition, since no differences between the CDR 0.5 and CDR 1 groups were observed, these groups were combined to form the mild AD group and were then compared to the CDR 0 group. ELISA data that display CDR 0.5 and CDR 1 separately can be found in FIG. 6. For reference, the concentrations of Aβ42 and total tau (which have been obtained previously to this study) in these CSF samples were included (FIG. 7).

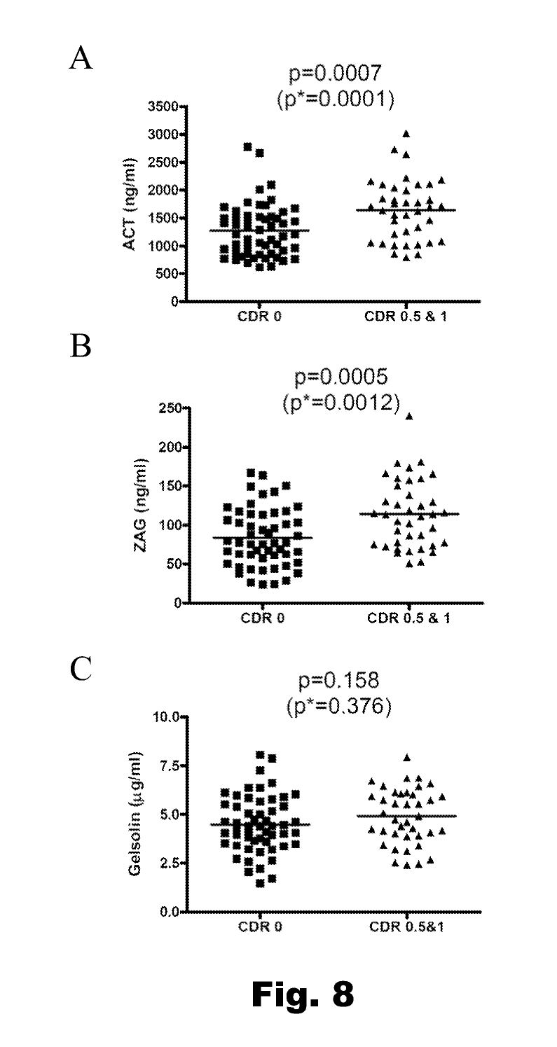

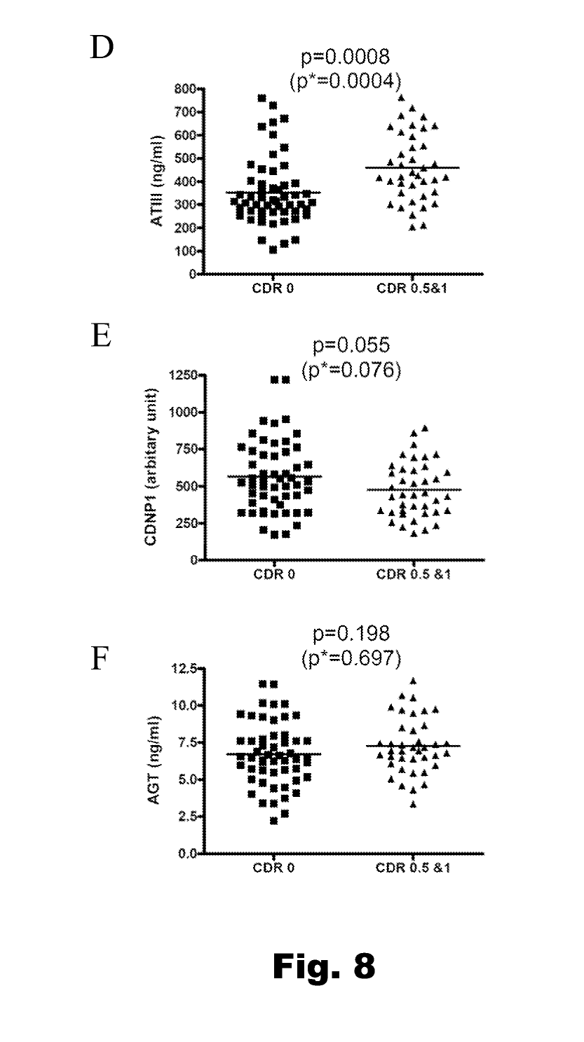

As shown in FIG. 8, the CSF levels of ACT, ATIII and ZAG are significantly increased in the very mild/mild AD group, confirming the 2D-DIGE findings. There is a trend for a decrease in CNDP1 levels in the very mild/mild AD group (FIG. 8), though this was not quite statistically significant (p=0.055). In contrast to the 2D-DIGE findings, a difference in levels of GSN or AGT between the two groups was not confirmed (FIG. 8). After log transformation (to approximate a normal distribution) the datasets were examined for any possible interactions between CSF concentrations of candidate biomarkers (including Aβ42 and total tau) and age/gender/number of APOE4 alleles. A significant correlation was found between the number of APOE4 alleles and CSF levels of Aβ42, total tau and AGT. There is also a positive correlation between age and CSF levels of ACT and GSN. In addition, an interaction between gender and CSF ZAG concentrations was observed, with males having higher levels than females. The initial observations based on the raw concentration data are not qualitatively different from those using the log-transformed and adjusted traits. The adjusted p values were included in FIG. 8 (designated by p*) for comparison. Since the differences in levels of GSN or AGT between the two groups were not confirmed, these two candidates were not included in subsequent analyses.

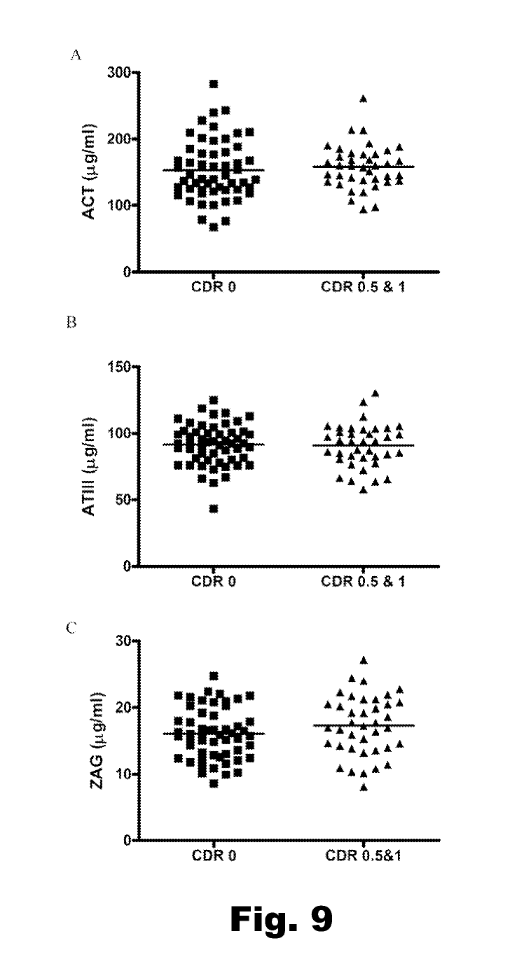

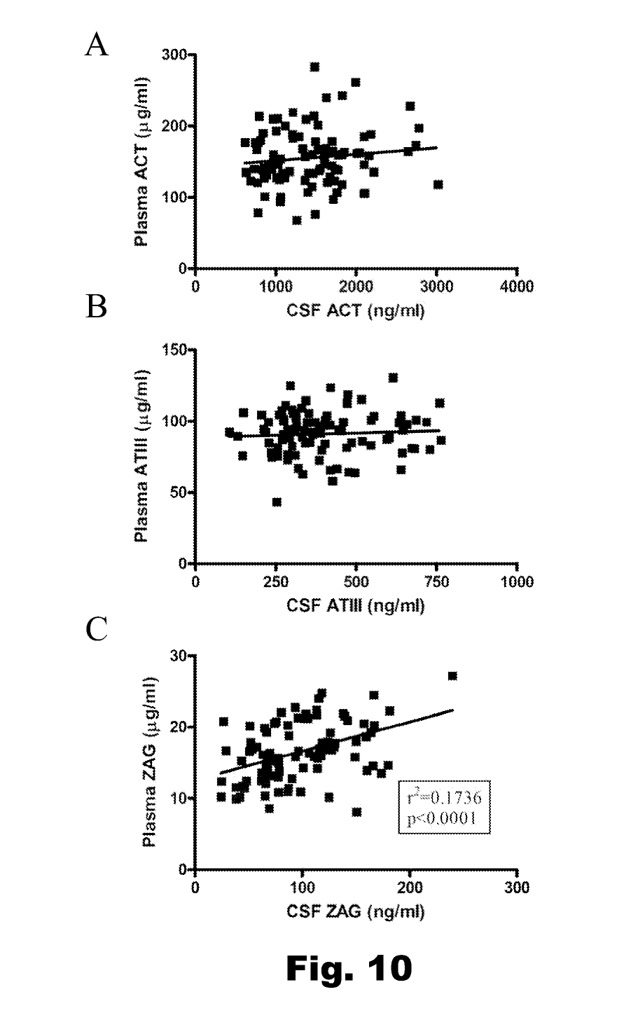

Next, whether similar differences exist in plasma as were found in CSF was assessed. Levels of ACT, ATIII and ZAG in paired plasma samples were not significantly different between the very mild/mild AD and control groups (FIG. 9). There was also no significant correlation between the CSF and plasma levels of ACT or ATIII, but there was a weak correlation between the CSF and plasma levels of ZAG (FIG. 10).

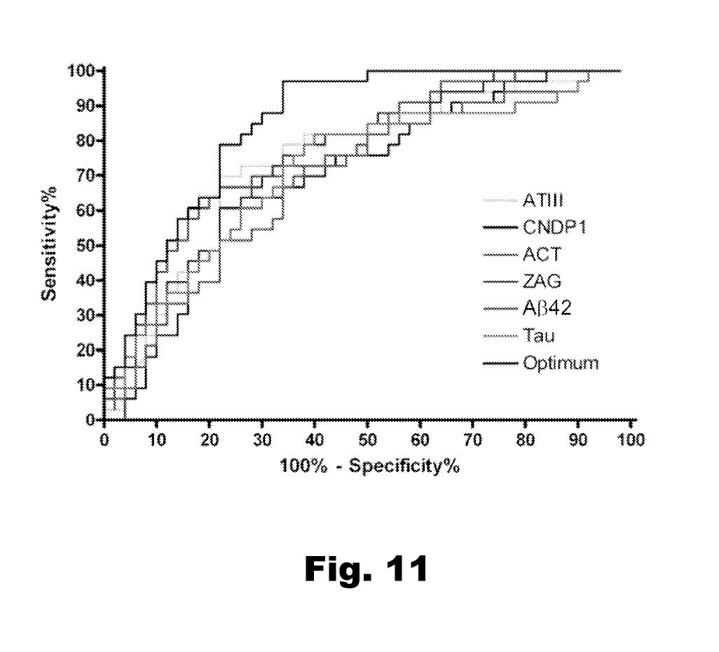

The relationships among the CSF concentrations of the candidate biomarkers (together with Aβ42 and tau) were examined (Table 4). None of the newly identified candidate biomarkers correlate with Aβ42 though ACT, CNDP1 and ZAG moderately correlate with tau. Interestingly, there are very strong, highly significant correlations between ACT, ATIII and ZAG (but not CNDP1) (Table 4). To preliminarily assess the potential use of these candidate biomarkers in differentiating subjects with very mild/mild AD from controls, a method proposed by Xiong et al. (Med Decis Making (2004) 24:659-69) was applied to determine the optimum linear combination of these biomarkers and calculated the confidence intervals on the AUC (the area under the receiver operating characteristic curve). Individual candidates by themselves are very comparable to those of Aβ42 and total tau in differentiating very mild/mild AD from controls (Table 5, FIG. 11). Importantly, when all biomarkers are optimally combined, this results in a higher AUC and sensitivity than for any single biomarker.

TABLE 4
Pearson Correlation Coefficients for the normalized, adjusted
biomarkers. Coefficients were derived using log-transformed data
that were also adjusted for interacting factors
(i.e. age, gender or the number of APOE4 allele).

| | ATIII | CNDP1 | ACT | ZAG | Aβ42 | TAU |
|---|---|---|---|---|---|---|
| ATIII | 1 | | | | | |
| CNDP1 | −0.007 | 1 | | | | |
| | (0.95) | | | | | |
| ACT | 0.73 | 0.030 | 1 | | | |
| | (<.0001) | (0.78) | | | | |
| ZAG | 0.66 | 0.026 | 0.71 | 1 | | |
| | (<.0001) | (0.81) | (<.0001) | | | |
| Aβ42 | 0.018 | 0.090 | −0.056 | 0.024 | 1 | |
| | (0.87) | (0.40) | (0.61) | (0.83) | | |
| TAU | 0.092 | 0.32 | 0.36 | 0.32 | −0.065 | 1 |
| | (0.40) | (0.003) | (0.0009) | (0.0024) | (0.55) | |

P-values are displayed in the parentheses.

TABLE 5
Comparison of the AUC of the optimum linear combination
(Optimum) to that of each individual biomarker (AUC: area
under the receiver operating curve).

| | | Sensitivity at 80% | |
|---|---|---|---|
| Marker | AUC (SE) | Specificity | p-value vs. Optumim |
| ATIII | 0.75 (0.10) | 0.48 | 0.29 |
| CNDP1 | 0.65 (0.12) | 0.24 | 0.099 |
| ACT | 0.75 (0.11) | 0.3 | 0.32 |
| ZAG | 0.72 (0.10) | 0.42 | 0.21 |
| Aβ42 | 0.64 (0.12) | 0.36 | 0.085 |
| tau | 0.68 (0.11) | 0.36 | 0.14 |
| Optimum | 0.88 (0.07) | 0.7 | — |

Cellular localization of ACT and ATIII. The cellular localization of some of the candidate biomarkers were examined in human and transgenic AD mouse brain. Consistent with previous reports (Abraham et al., 1998; Abraham et al., Neurobiol aging et al. (1990) 11:123-29), ACT is co-localized with amyloid plaques in human AD brain and is also present in neurons and astrocytes. Also, antibodies to ATIII label amyloid plaques and neurofibrillary tangles (NFT) in human AD brain, in accordance with a previous report (Kalatira et al, Am J Pathol (1993) 143:886-893).

### Discussion:

In this study, candidate biomarkers for mild AD were identified by analyzing a small group of CSF samples (mild AD vs. control) with a comparative proteomic approach. Importantly, selected biomarkers were validated with ELISA using a larger sample set. Among the identified biomarkers is ACT, which has been identified previously in other reports. Novel biomarkers such as ATIII, ZAG and CNDP1 were also identified, whose roles as AD biomarkers in CSF have not been previously assessed and validated. This is the first proteomic study on AD biomarkers that not only discovered candidate protein biomarkers, but also validated these candidates in a much larger and independent sample set with a sensitive and quantitative method (i.e. ELISA) in multiple, well characterized individuals. Thus, this proof-of-principle study demonstrates that this proteomic approach can be used to reliably identify AD biomarkers in CSF.

The proteomic approach taken included multi-affinity depletion, 2-D DIGE, and nano-LC-FTMS. The sensitive, high resolution mass spectrometric resulted in the identification in 16 of the 18 gel features that showed significant differential abundances between the two CDR groups. The overall result was the generation of reliable data, which is demonstrated by the low false discovery rate (FDR) in the study. FDR is defined as the number of expected false predictions divided by the number of total predictions, which in the study is 21. The number of expected false predictions was estimated using a previous set of CSF proteomic data (generated using the same approach and performed in the same lab by the same people). A “same-same” comparison was approximated by comparing a set of 6 CSF samples from 6 individuals to a second set of 6 CSF samples from the same individuals, taken 2 weeks apart. Any differentially expressed proteins found in such a comparison would be false positives because in the ideal scenario, there should be no differences detected in a same-same comparison. Interestingly, no protein spots (0 out of 306 matched spots) were found that were significantly different between the two groups. With a close-to-zero FDR when the p value cutoff is set at 0.05, the p value cutoff could potentially be increased to maximize the number of biomarker candidates discovered while still maintaining a low FDR.

Four of the 6 selected biomarker candidates were validated using ELISA with a much larger characterized sample set. Although protein spots that correspond to GSN and AGT display differential abundance in 2D-DIGE, a difference between the mild AD and control group via ELISA was not detected. One possible explanation is the existence of different isoforms for a single protein. GSN has two isoforms “a” and “b” which differ only by a stretch of 24 amino acids at the N terminus. The unique peptides for the two isoforms were not detected in the MS data and the ELISA is not isoform specific. AGT is known to have different isoforms due to glycosylation Matsubara et al., Ann Neurol (1990) 28:561-67). If the 2D-DIGE observations only reflect differences in certain isoforms, it might not result in a difference in total protein level measured by ELISA. Thus, to the extent that AD is associated with changes in the level of specific protein isoforms, ELISA for total protein levels might not detect such changes.

One of the validated candidate biomarkers, ACT, has been previously proposed as a possible biomarker for AD. An increase in CSF ACT level was observed in the AD group, in agreement with a number of previous studies (Matsubara et al., Ann Neurol (1990) 28:561-67; DeKosky et al., Ann Neurol (2003) 53:81-90; Harigaya et al., Intern Ned (1995) 34:481-84; Licastro et al., Alzheimer Dis Assoc Disord (1995) 9:112-118). There was no significant change in ACT levels in plasma in the dementia group nor a correlation between the CSF and plasma levels of this protein. This is similar to some previous findings [33, 36-39] (Matsubara et al., Ann Neurol (1990) 28:561-67; Licastro et al., Alzheimer Dis Assoc Disord (1995) 9:112-118; Pirttila et al., Neurobiol Aging (1994) 15:313-317; Licastro et al., Dement Geriatr Cogn Disord (2000) 11:25-28; Lanzrein et al., Alzhemier Dis Assoc Disord (1998) 12:215-227) but not others (Matsubara et al., Ann Neurol (1990) 28:561-67; DeKosky et al., Ann Neurol (2003) 53:81-90; Licastro et al., Alzheimer Dis Assoc Disord (1995) 9:112-118; Oishi et al., Ann Clin Lab Sci (1996) 26:340-45; Licastro et al., J Nuroimmunol (2000) 103:97-102). The ACT index (a calculated quotient using the ACT and albumin content of CSF and plasma to detect intrathecal ACT synthesis) was also measured and it was found that 90% of the samples have an index above 1, with an average index of 2.4. These results indicate independent within-CNS production as the main source of ACT in CSF.

In contrast to ACT, other validated candidates (i.e. ATIII, ZAG and CNDP1) have not been well-studied in AD and therefore represent novel potential biomarkers. ATIII, like ACT, is a serine protease inhibitor. In the present and another study (Kalaria et al., Am J Pathol (1993) 143:886-893), ATIII was shown to localize to both amyloid plaques and NFT in AD brain. However, ATIII has not previously been reported to be increased in AD CSF. It is not clear why ZAG and CNDP1 are altered in AD CSF or their potential roles in AD pathogenesis. ZAG is a soluble glycoprotein and present in a variety of body fluids (Poortmans et al., J Lab Clin Med (1968) 71:807-11). The biological functions of ZAG are largely unknown. One 2D-gel-based proteomic study identified ZAG as one of the proteins that was increased in AD CSF (Davidsson et al., Neuroreport (2002) 13:611-15) though this was not validated in a large sample set. Carnosinase I (CNDP1) hydrolyzes carnosine (β-alanlyl-L-histidine) and homocarnosine (γ-aminobutyryl-L-histidine), both of which are believed to be neuro-protective (Teufel et al., J Biol Chem (2003) 278:6521-31). Decreased serum levels of CNDP1 have been found in patients with Parkinson's disease, multiple sclerosis and in patients after a cerebrovascular accident (Wassif et al., Clin Chim Acta (1994) 225:57-64).

The degree of the changes of these validated biomarker candidates (in AD CSF) is moderate (within 30%), with overlaps between the AD and control group. However, the magnitude of the difference in levels of ACT, ATIII and ZAG is comparable to that of Aβ42, currently one of the best CSF biomarkers of AD that correlates very well with the presence or absence of amyloid in the brain (Fagan et al., Ann Neurol (2006) 59:512-19). Importantly, changes in these biomarkers are already evident in the very mildly demented CDR 0.5 group (FIG. 7), suggesting the suitability of these candidates for the early detection of AD. Due to the existence of preclinical AD in cognitively normal individuals, the CDR 0 group is likely heterogeneous, containing subjects with and without AD pathology in the brain. Indeed, a recent study demonstrated the presence of brain amyloid and low levels of CSF Aβ42 in a subset of cognitively normal individuals (Fagan et al., Ann Neurol (2006) 59:512-19). Such preclinical AD pathology might in part account for the overlap between AD and control group in terms of biomarker concentrations.

Statistical analyses demonstrate that the biomarker candidates identified have diagnostic power/accuracy comparable to that of Aβ42 and total tau. When all biomarkers are combined, a greater AUC and sensitivity can be achieved. Although the difference is not statistically significant in this study (including 50 CDR 0 subjects and 33 mild AD subjects), this trend may become significant when larger sample sets are assessed.

### Example 3

### Materials and Methods for Example 3

Subjects: There are three cohorts used in this example.

Discovery cohort: Subjects (N=48), community-dwelling volunteers from University of Washington [N=18], Oregon Health and Science University [N=11], University of Pennsylv ania [N=11], and University of California San Diego [N=8], and were 51-87 years of age and in good general health, having no other neurological, psychiatric, or major medical diagnoses that could contribute importantly to dementia, nor use of exclusionary medications within 1-3 months of lumbar puncture (LP) (e.g. neuroleptics, anticonvulsants, anticoagulants). Study protocols at each institution were approved by their respective Institutional Review Boards and written informed consent was obtained from each participant. Cognitive status was evaluated based on criteria from the National Institute of Neurological and Communicative Diseases and Stroke-Alzheimer's Disease and Related Disorders Association. CSF was collected in the morning by LP after overnight fasting and immediately frozen at −80° C. Subjects with a clinical dementia rating (CDR) of 0 (N=24), indicating no dementia, and CDR 1 (N=24), indicating mild dementia, were selected from a larger group of 120 samples on the basis of CSF Aβ42 (relatively high and low values, respectively), and, when possible, CSF tau (relatively low and high values, respectively) to increase the likelihood of CDR 1 subjects having and CDR 0 subjects not having AD pathology. CSF Aβ42 and tau levels were measured in a single laboratory using well-established ELISAs (Innotest, Innogenetics). Quantitative thresholds were not defined prior to sample selection; the lowest CDR 0 and highest CDR 1 CSF Aβ42 value were 572 and 399 pg/mL, respectively; CSF tau ranges were CDR 0: 141-448 pg/mL, CDR 1: 216-1965 pg/mL.

Validation cohort: Subjects (N=292), community-dwelling volunteers enrolled in longitudinal studies of healthy aging and dementia at the Washington University Alzheimer Disease Research Center (WU-ADRC), were 60 years of age and met the same exclusion criteria as the discovery cohort. The study protocol was approved by the Human Studies Committee at WU, and we obtained written and verbal informed consent from participants at enrollment. CDR status was determined as with the discovery cohort, with an additional category of CDR 0.5, indicating very mild dementia; some of these met criteria for MCI and some were more mildly impaired, or “pre-MCI”. A subset of subjects (N=159) underwent positron emission tomography (PET) imaging with Pittsburgh Compound-B (PIB) for assessment of in vivo amyloid burden. Apolipoprotein E (APOE) genotypes were determined by the WU-ADRC Genetics Core. Fasted CSF was collected, mixed, centrifuged, and frozen at −80° C. in polypropylene tubes; blood was collected at the time of LP, and plasma prepared by centrifugation and stored at −80° C.

FTLD/PSP Cohort: Volunteer subjects were diagnosed with frontotemporal lobar degeneration (FTLD) (N=9) or progressive supranuclear palsy (PSP) (N=6) at the University of California San Francisco (UCSF) Memory and Aging Center using published criteria. Subjects in the FTLD group met criteria for one of the three clinical syndromes that comprise FTLD: frontotemporal dementia (FTD) (N=6), semantic dementia (SD) (N=1), and progressive non-fluent aphasia (PNFA) (N=2). The study protocol was approved by the UCSF Committee on Human Research, and informed consent was obtained from all participants. CSF was collected by LP and immediately frozen at −80° C.

2-D DIGE LC-MS/MS Proteomic Analysis: Briefly, discovery cohort CSF samples and a pooled reference sample were immunodepleted of six highly abundant proteins (albumin, IgG, α1-antitrypsin, IgA, haptoglobin, transferrin). Samples were randomly paired (CDR 0 and CDR 1), labeled with one of three cyanine dyes, and loaded with the labeled reference sample onto the same 2-D gel. Protein spot quantification and between-gel spot matching were performed on digitized images. To focus efforts on candidate biomarkers more likely to be measurable in the CSF of a majority of individuals, only gel features with significant intensity differences between CDR 0 and CDR 1 groups (Student's t-test, α=0.05) that were present in >50% of gels were excised, trypsinized, and subjected to LC-MS/MS. Proteins were identified from peptide fragmentation spectra using MASCOT (v2.8, Matrix Sciences) and the NCBI non-redundant protein database (downloaded Nov. 11, 2008).

Enzyme Linked Immunosorbent Assays (ELISAs): CSF and plasma samples were analyzed by ELISA for Aβ42, total tau, and phospho-tau181 (Innotest, Innogenetics) after one freeze-thaw, and for YKL-40 (Quidel) after two freeze-thaw cycles. Intra- and inter-assay coefficient of variation for CSF YKL-40 were 5.27% and 6.03%, respectively; for plasma, 5.73% and 11.26%.

Statistical Analyses: Correlations were evaluated using the Pearson rho correlation coefficient (α=0.05). Survival analyses assessed the ability of baseline biomarkers and biomarker ratios to predict time to conversion from cognitive normalcy (CDR 0) to very mild or mild dementia (CDR 0.5, 1) and time to progression from very mild dementia (CDR 0.5) to more severe dementia (CDR>0.5). Data from subjects who did not convert/progress were statistically censored at the date of last assessment. Biomarker measurements were converted to standard Z-scores to allow comparison of hazard ratios between different biomarkers. Cox proportional hazard models adjusted for age and gender were conducted treating the CSF biomarkers as continuous and categorical variables. Categorical analyses compared subjects within the highest tertile of baseline values to those within the lowest two tertiles; this tertile-based assessment was applied because Kaplan-Meier curves illustrating the unadjusted time to CDR>0 for each tertile of each biomarker suggested similar outcomes for the lower two tertiles. The difference between the survival curves reflecting the upper tertile versus the lower tertiles of each biomarker was tested using the log-rank test. Survival analyses were conducted using baseline CDR scores determined at clinical assessment prior to LP; analyses using scores determined at clinical assessment closest to LP yielded almost identical results. Similar survival analyses were carried out for plasma YKL-40.

Immunohistochemistry: Six-βm-thick sections of formalin-fixed, paraffin-embedded human postmortem brain tissue (middle frontal gyrus, post mortem interval <6 hrs) from the WU-ADRC Neuropathology Core were double-labeled using rabbit anti-human YKL-40 antibody (Quidel) in series with either goat anti-human GFAP (Santa Cruz), mouse anti-human HLA Class II antigen, LN-3 (Novocastra), RCA-1 (Vector), or mouse anti-human PHF-1 (gift of Dr. Peter Davies), followed by staining with the ImmPress kit (Vector). In control experiments, the primary antibody was omitted and replaced with 1% bovine serum albumin-PBS. Thioflavin S stain (1% aqueous) was applied for 20 minutes and destained with 50% ethanol.

### Results:

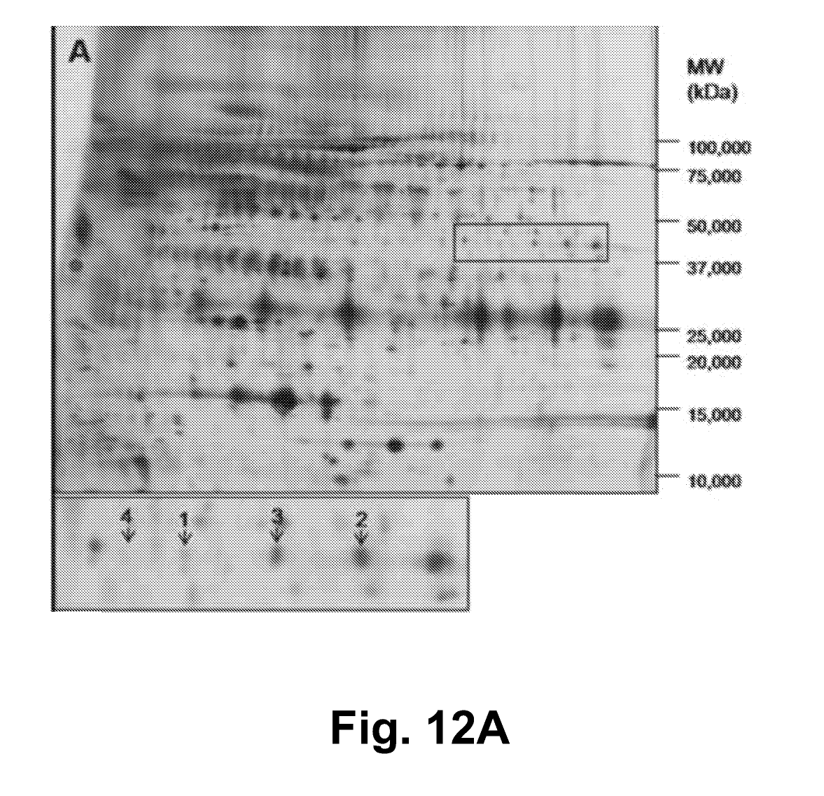

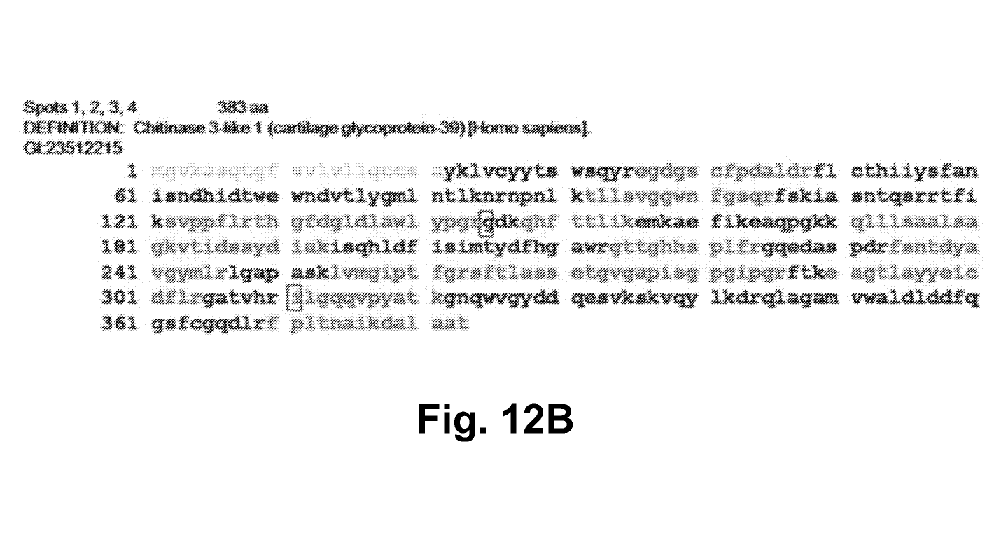

Proteomic Analysis Identifies YKL-40 as Increased in AD CSF: To identify new candidate biomarkers for AD, we utilized an unbiased proteomics approach, 2-D DIGE LC-MS/MS, to compare the concentrations of CSF proteins in individuals with mild dementia (CDR 1, N=24) of the Alzheimer s type to those in individuals without dementia (CDR 0, N=24). The two groups differed with respect to age at LP and gender (CDR 0: 64.8 yrs, 38% female; CDR 1: 72.8 yrs, 54% female). From this proteomic analysis, we identified 47 proteins that differed in abundance between the CDR 0 and CDR 1 groups (unpublished data); one of the most promising, in terms of fold-change and novelty, was YKL-40. Interestingly, in a smaller, previous study, we identified YKL-40 as being significantly more abundant in CSF from CDR 0.5 relative to CDR 0 subjects. YKL-40 appeared in four gel features that were more abundant in the CDR 1 group (FIG. 12A). Tryptic peptides from these spots collectively provide amino acid sequence coverage of 52% and span virtually the full length of the protein (FIG. 12B), suggesting that these spots represent full-length secreted YKL-40. We hypothesize that this pattern of four spots may be due to allelic differences, post-translational modifications, or both.

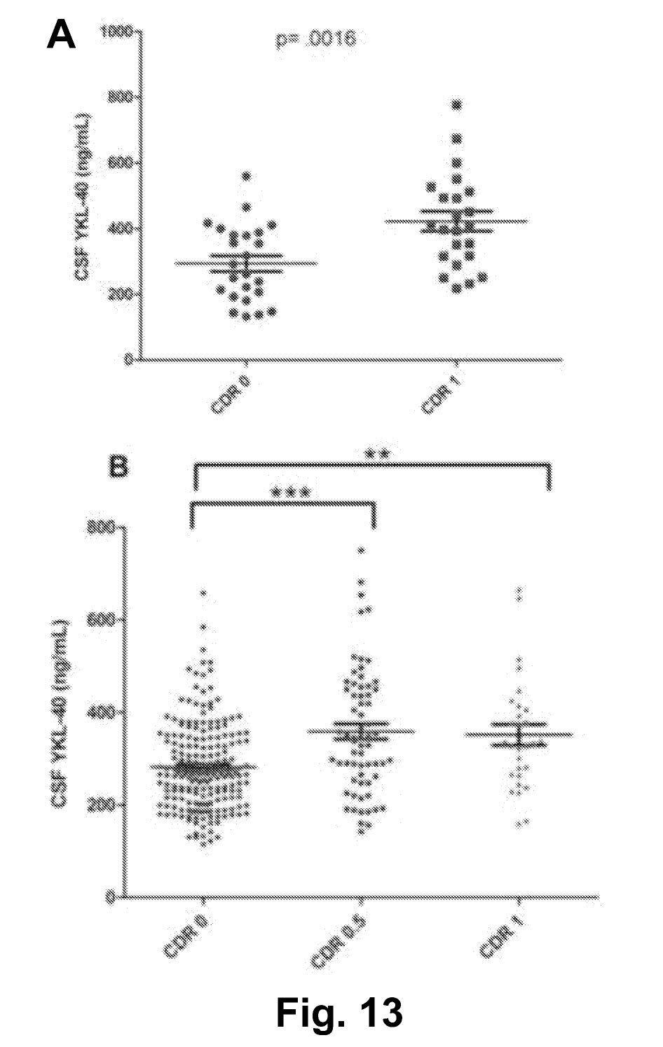

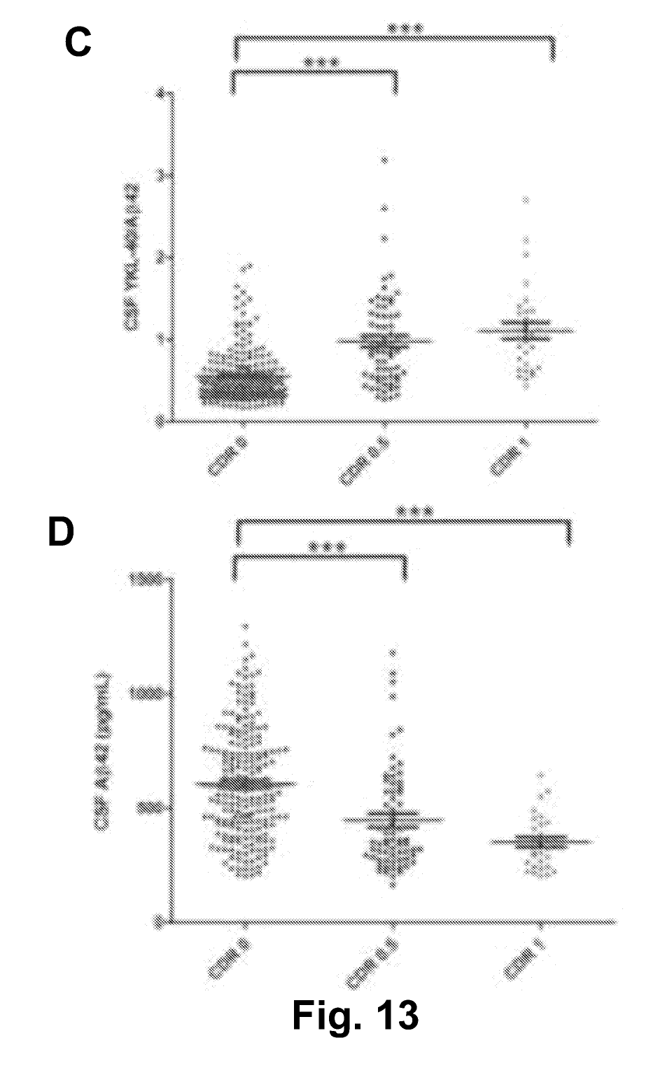

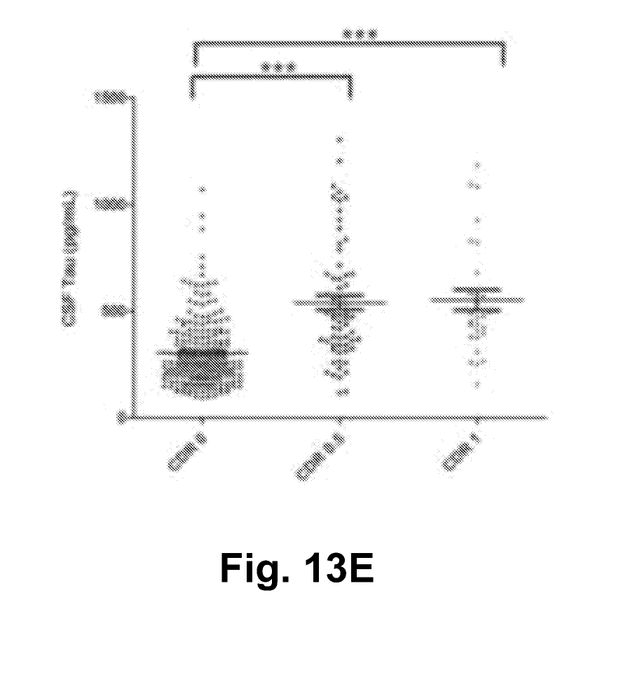

ELISA Confirms Increased CSF YKL-40 in AD in Original and Independent Cohorts To validate the 2-D DIGE findings, a YKL-40 ELISA was applied to the original “discovery” cohort samples (one sample was unavailable for re-evaluation, N=47). Mean CSF YKL-40 was increased 43% in the CDR 1 vs CDR 0 group (p=0.0016) (FIG. 13A), consistent with the foldchanges measured by 2-D DIGE. We next assayed a larger, independent set of CDR 0, 0.5, and 1 CSF samples collected at the WU-ADRC(N=292) that was not preselected on the basis of CSF Aβ42 and tau values (characteristics at baseline assessment in Table 6). In this validation cohort, mean CSF YKL-40 was significantly (27%) higher in the CDR 0.5 and CDR 1 groups vs. CDR 0 (p<0.0001 and p=0.004, respectively) (FIG. 13B). An analysis of covariance (ANCOVA) revealed that this increase remained significant after adjusting for age, F(2, 288)=9.075, p<0.0001.

TABLE 6
Demographic, Clinical, and Genotypic Characteristics of Validation
Cohort

| Characteristic | CDR 0 | CDR 0.5 | CDR 1 |
|---|---|---|---|
| n | 198 | 65 | 29 |
| Gender (% Female) | 63% | 54% | 52% |
| APOE genotype, % ε4+ | 35% | 51% | 59% |
| Mean MMSE score (SD) | 28.9 (1.3) | 26.3 (2.8) | 22.3 (3.9) |
| Mean age at LP (SD), yrs | 71.0 (7.3) | 73.8 (6.8) | 76.5 (6.2) |
| Mean CSF Aβ42 (SD), | 605 (240) | 446 (230) | 351 (118) |
| pg/mL | | | |
| Mean CSF tau (SD), pg/mL | 304 (161) | 539 (276) | 552 (263) |
| Mean CSF ptau181 (SD), | 55 (25) | 85 (44) | 77 (38) |
| pg/mL | | | |

Abbreviations:
CDR, Clinical Dementia Rating;
APOE, apolipoprotein E;
MMSE, Mini-Mental State Examination;
LP, lumbar puncture;
SD, standard deviation;
CSF, cerebrospinal fluid;
Aβ-42, amyloid-beta peptide 1-42;
ptau181, tau phosphorylated at threonine 181

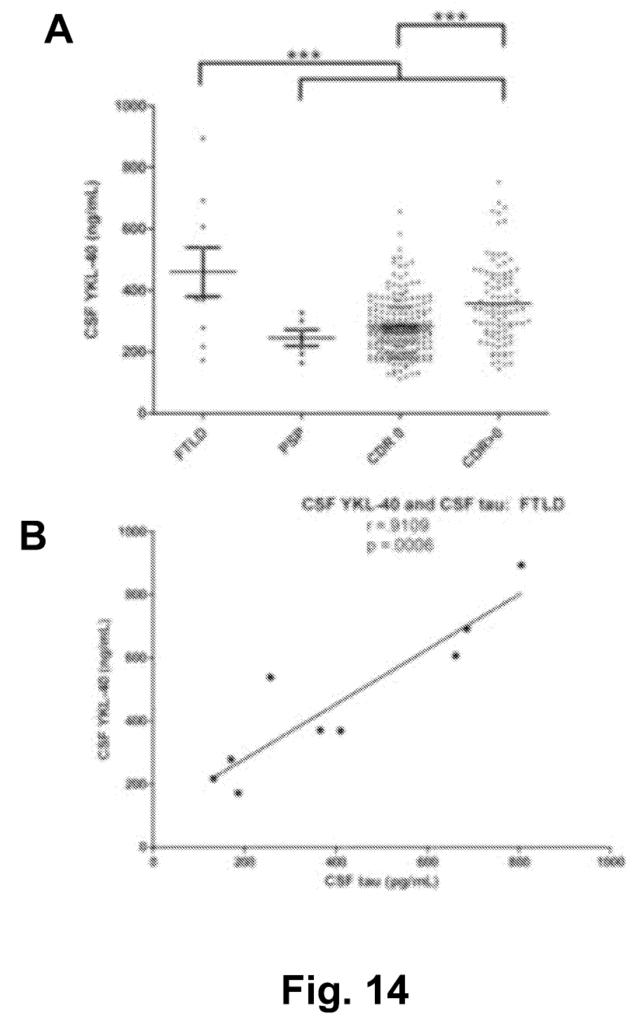

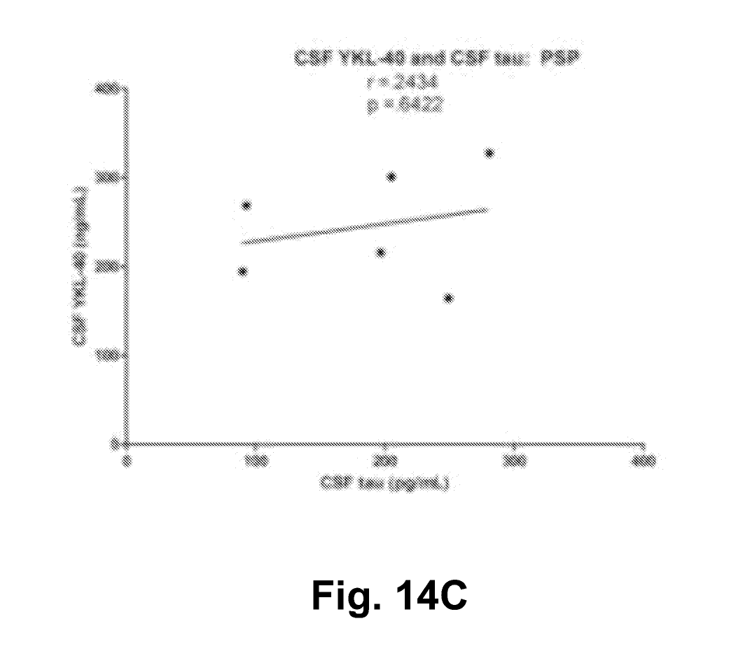

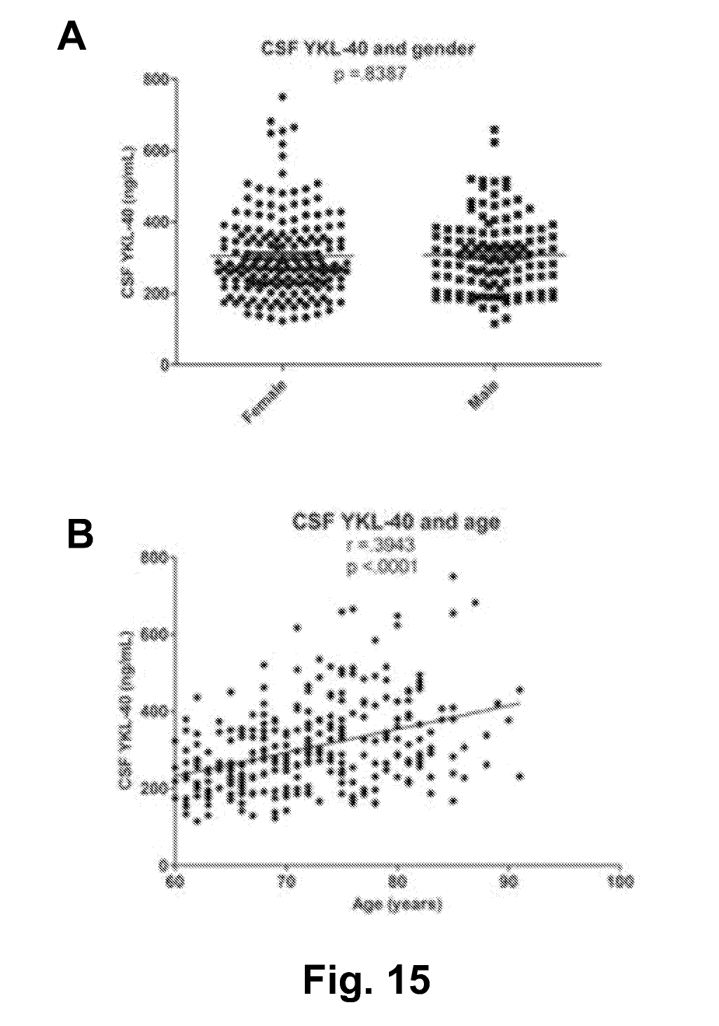

CSF YKL-40 is Increased in FTLD and Decreased in PSP: In an effort to determine whether CSF YKL-40 might have potential to distinguish AD from other dementing illnesses, we evaluated levels in two other neurodegenerative diseases: frontotemporal lobar degeneration (FTLD, N=9) and progressive supranuclear palsy (PSP, N=6). Mean CSF YKL-40 was increased in FTLD relative to AD, although a wide range of values was observed, possibly reflecting the pathological heterogeneity of FTLD; in contrast, PSP cases showed relatively low levels and range of CSF YKL-40 (FIG. 14A). This study thus showed that CSF YKL-40 is useful to distinguish AD from some other forms of neurodegenerative disease.

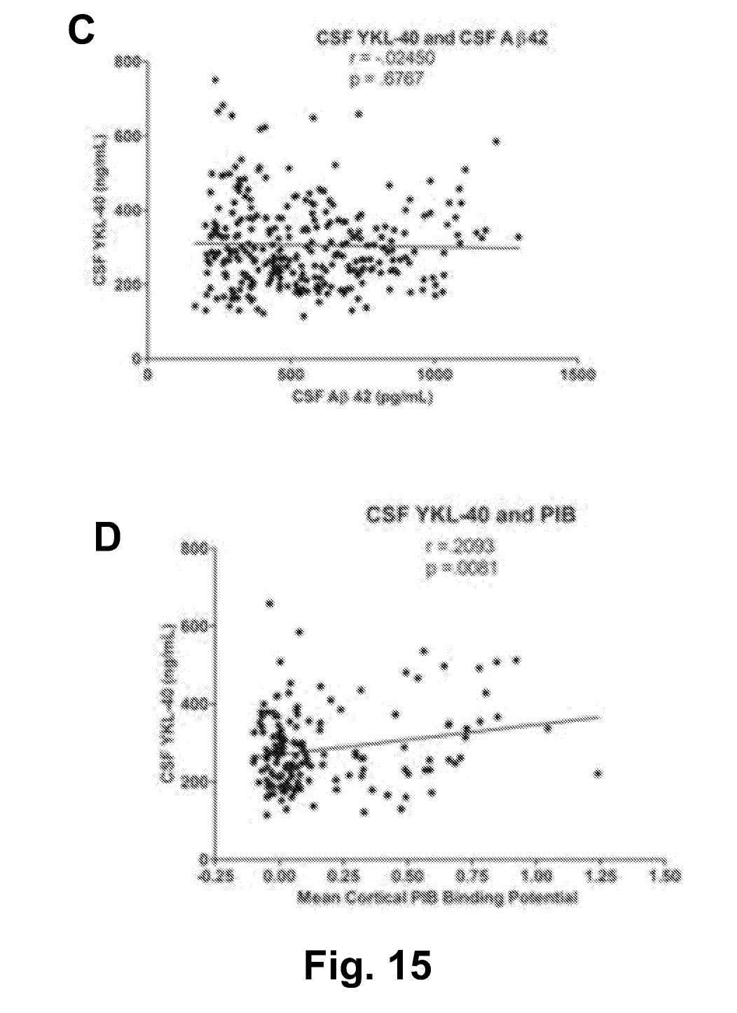

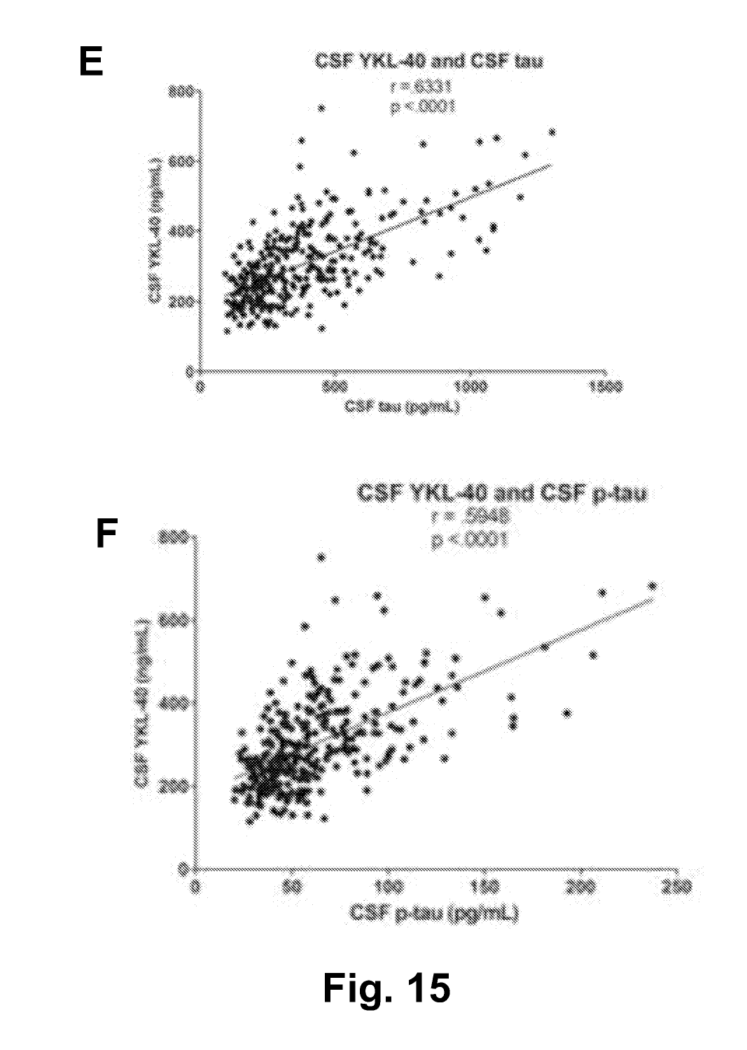

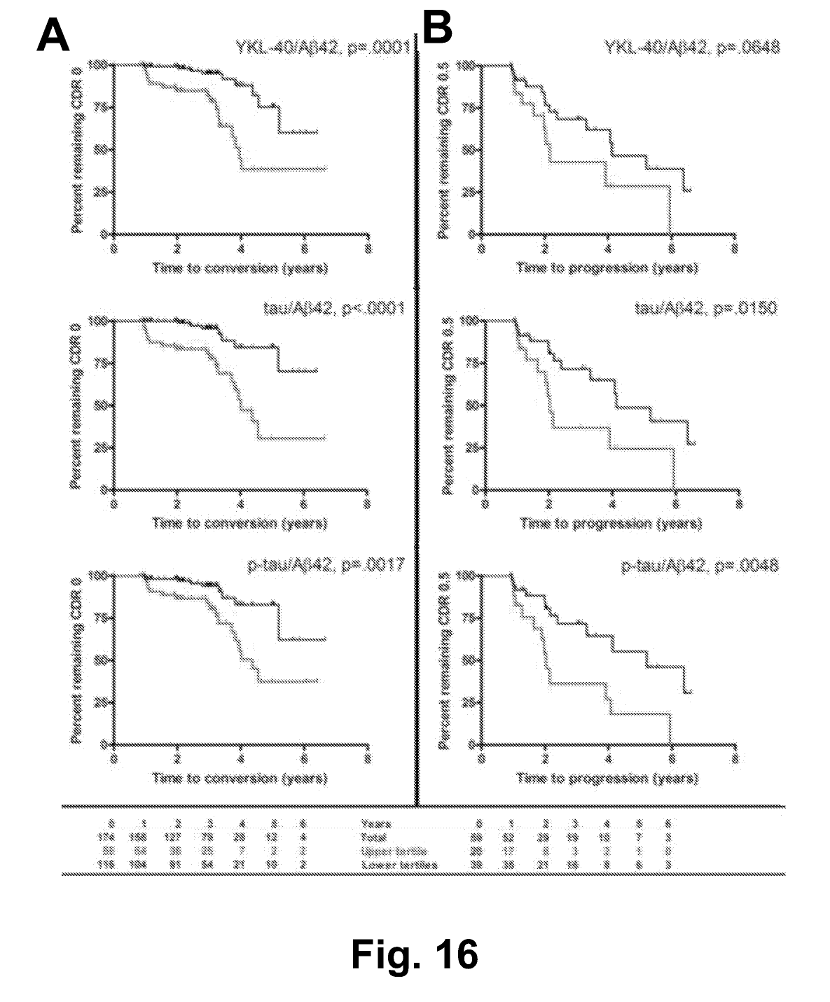

Correlation of CSF YKL-40 With Demographic Features and Other Biomarker Values: Because the CDR 0, 0.5, and 1 groups show somewhat different distributions with regard to age at LP, gender, and APOE genotype, levels of CSF YKL-40 were evaluated for potential correlation with these variables. CSF YKL-40 levels did not vary based on gender (p=0.8355) or APOE genotype (not shown) but did correlate with increasing age (r=0.3943, p<0.0001) (FIG. 15). Next, seeking insight into the role of YKL-40 in AD pathology, we evaluated its associations with CSF Aβ42, CSF tau, and cortical amyloid burden measured by PIB-PET imaging. In this validation cohort, CSF YKL-40 did not correlate with CSF Aβ42 (r=−0.02463, p=0.6745), but did correlate with CSF tau (r=0.6331, p<0.0001), and p-tau181 (r=0.5947, p<0.0001), and modestly with cortical amyloid burden (r=0.2093, p=0.0081) (FIG. 15). Further, a similar correlation of CSF YKL-40 with tau was observed in FTLD (r=0.9109, p=0.0006), but not in PSP (r=0.2434, p=0.6422) (FIGS. 14B and C). Therefore, these two biomarkers are not inextricably linked, and that they may reflect separate but interrelated pathophysiological processes.

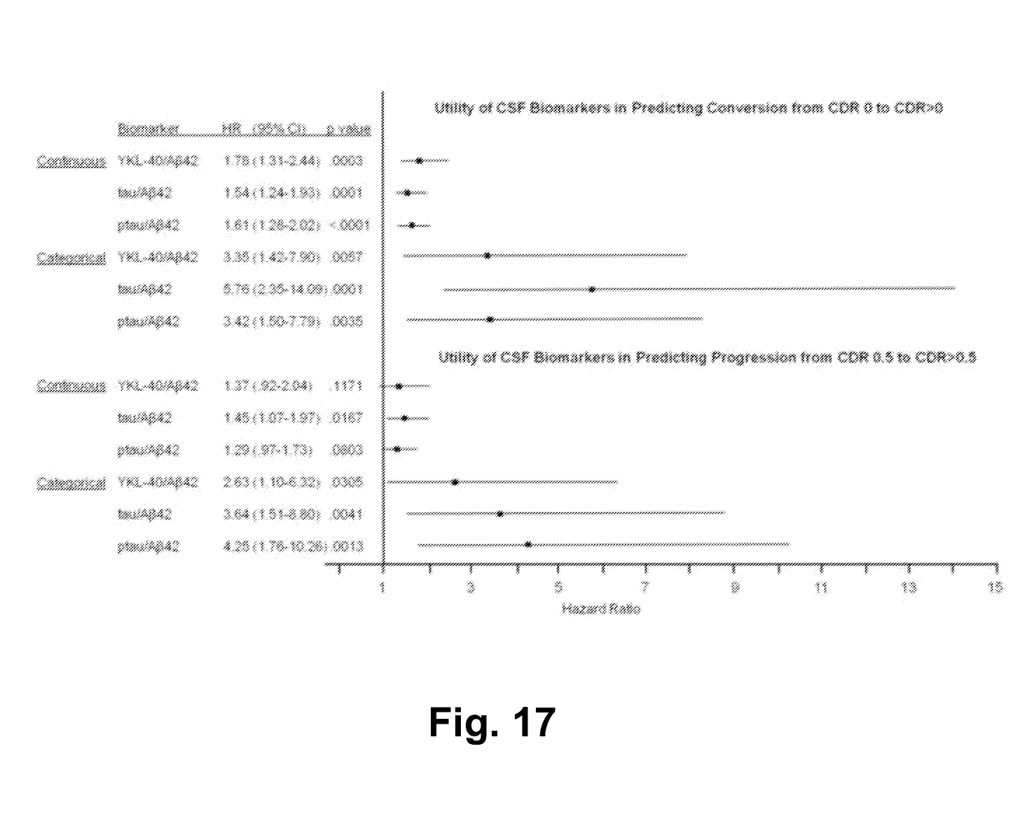

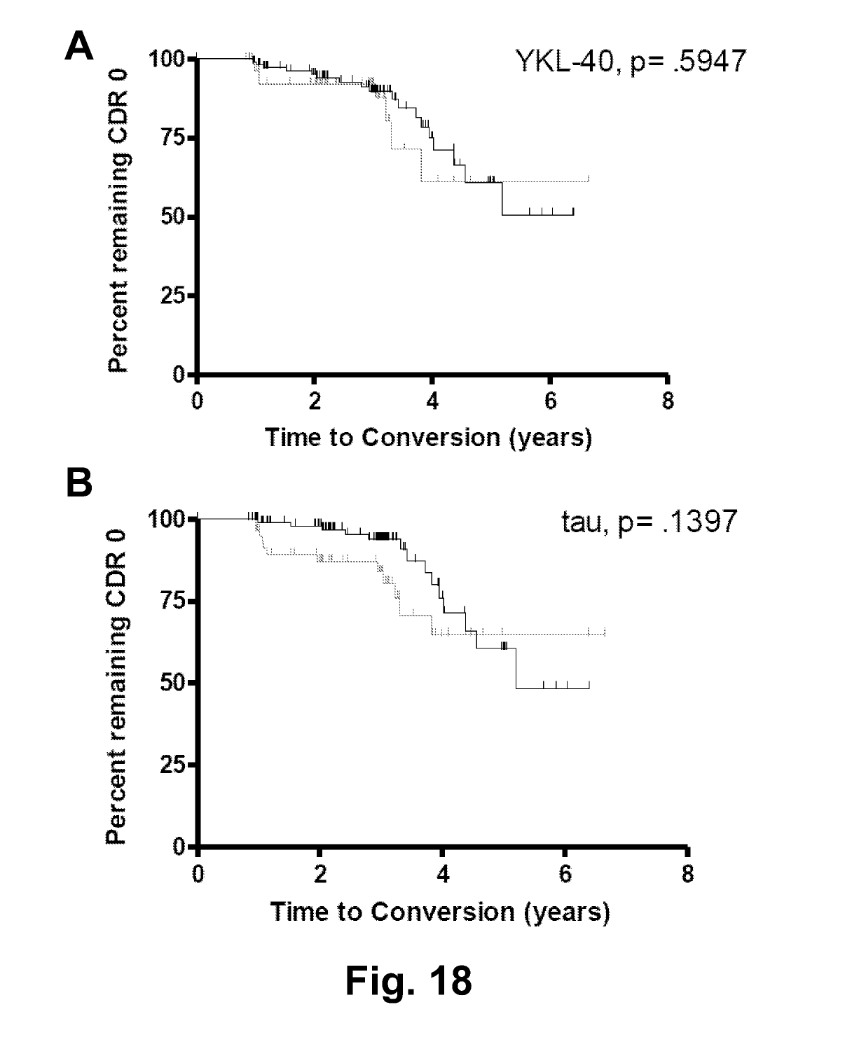

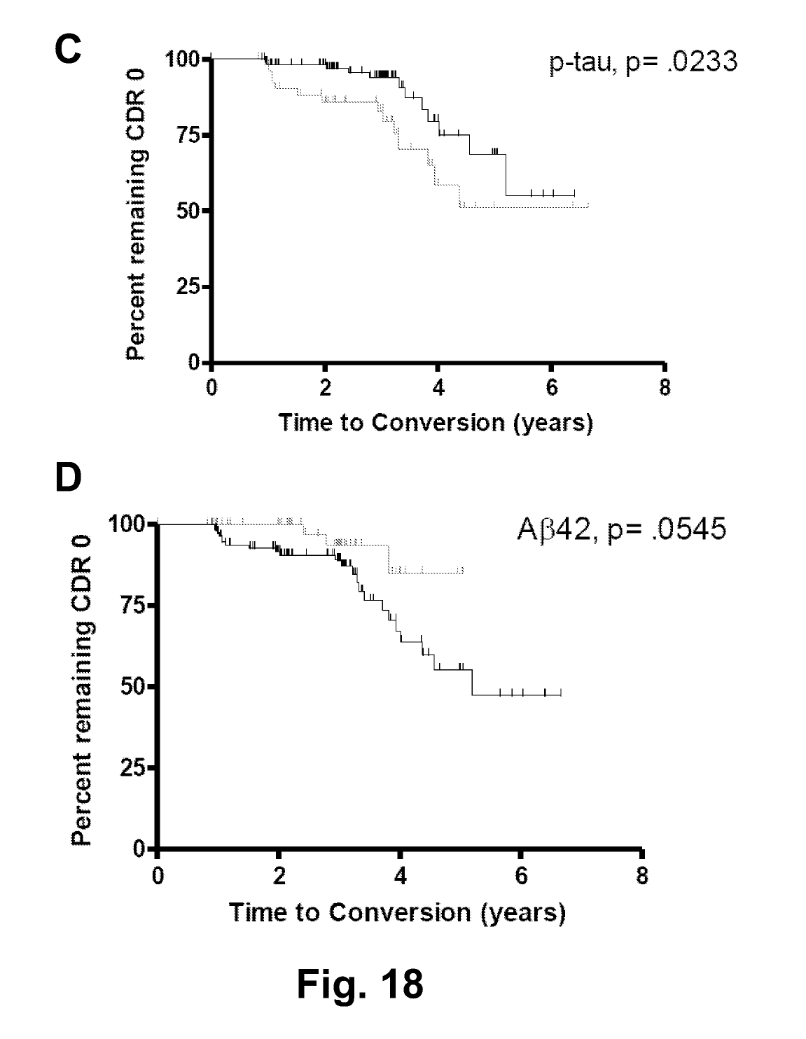

Ability of CSF YKL-40 To Predict Onset and Progression of Dementia: Recognizing the need for preclinical diagnosis and prognosis, we applied survival analyses to evaluate whether CSF YKL-40 can predict risk of developing cognitive impairment (conversion from CDR 0 to CDR>0) and of dementia progression (CDR 0.5 to CDR>0.5). Of the 174 CDR 0 subjects with at least one follow-up clinical assessment, 26 received a CDR>0 at follow-up, and thus were classified as “converters.” Since CSF tau/Aβ42 and p-tau181/Aβ42 ratios have been shown to predict cognitive decline in cognitively normal and MCI cohorts, survival analyses were also conducted for these biomarkers. Treated as categorical variables, subjects with high ratios (upper tertile) of CSF YKL-40/Aβ42, tau/Aβ42, and p-tau181/Aβ42 were faster to convert to CDR>0 than were subjects with lower ratios (lower tertiles) (FIG. 16A), even after adjusting for age and gender (FIG. 17, Table 7A and B). Likewise, when treated as continuous variables, CSF YKL-40/Aβ42, tau/Aβ42, and p-tau181/Aβ42 ratios again predicted conversion from CDR 0 to CDR>0 (p=0.0003, p=0.0001, p<0.0001, respectively) after adjustment for age and gender (FIG. 17, Table 7A and B). Importantly, when evaluated individually, CSF YKL-40, Aβ42, tau, and p-tau181 did not perform as well as the YKL-40/Aβ42, tau/Aβ42, and p-tau181/Aβ42 ratios at predicting conversion from CDR 0 to CDR>0 (FIG. 18, Table 8). Thus, the CSF YKL-40/Aβ42 ratio, as a prognostic biomarker of future cognitive impairment in normal individuals, is comparable to the best CSF biomarkers of this type to date, tau/Aβ42 and p-tau181/Aβ42. Of the 59 CDR 0.5 subjects with at least one follow-up clinical assessment, 24 received a CDR>0.5 at follow-up, and thus were classified as “progressors.” Kaplan-Meier estimates of the rate of progression suggest that those with high CSF YKL-40/Aβ42 ratios (upper tertile) were faster to progress to CDR>0.5 than those with lower CSF YKL-40/Aβ42 ratios (lower two tertiles) (p=0.0648) (FIG. 16B). The tau/Aβ42 and p-tau181/Aβ42 ratios showed similar patterns (FIG. 16B). After adjustment for age and gender, similar results were found for all three categorical biomarker variables (FIG. 17, Table 7 A and B). Treated as a continuous variable and adjusted for age and gender, p-tau181/Aβ42 and YKL-40/Aβ42 ratios showed trends associated with time to progression that did not reach statistical significance (FIG. 17, Table 7 A and B).

TABLE 7A
Utility of CSF Biomarkers in Predicting Conversion from CDR 0 to CDR > 0

| | YKL-40/Aβ42 | | | tau/Aβ42 | | | ptau/Aβ42 | | |
|---|---|---|---|---|---|---|---|---|---|
| | | | p | | | p | | | p |
| | HR | 95% CI | value | HR | 95% CI | value | HR | 95% CI | value |
| Biomarker- | 1.78 | 1.31-2.44 | .0003 | 1.54 | 1.24-1.93 | .0001 | 1.61 | 1.28-2.02 | <.0001 |
| continuous | | | | | | | | | |
| Age, yr | 1.05 | 0.99-1.10 | .0844 | 1.07 | 1.01-1.12 | .0177 | 1.06 | 1.01-1.12 | .0181 |
| Women | 0.53 | 0.24-1.18 | .1196 | 0.50 | 0.22-1.14 | .1003 | 0.56 | 0.25-1.26 | .1596 |
| Biomarker- | 3.35 | 1.42-7.90 | .0057 | 5.76 | 2.35-14.09 | .0001 | 3.42 | 1.50-7.79 | .0035 |
| categorical | | | | | | | | | |
| Age, yr | 1.05 | 0.99-1.10 | .0950 | 1.07 | 1.02-1.13 | .0116 | 1.06 | 1.01-1.12 | .0194 |
| Women | 0.71 | 0.31-1.63 | .4181 | 0.66 | 0.29-1.49 | .3123 | 0.65 | 0.28-1.50 | .3110 |

TABLE 7B
Utility of CSF Biomarkers in Predicting Progression from CDR 0.5 to CDR > 0.5

| | YKL-40/Aβ42 | | | tau/Aβ42 | | | ptau/Aβ42 | | |
|---|---|---|---|---|---|---|---|---|---|
| | | | p | | | p | | | p |
| | HR | 95% CI | value | HR | 95% CI | value | HR | 95% CI | value |
| Biomarker- | 1.37 | 0.92-2.04 | .1171 | 1.45 | 1.07-1.97 | .0167 | 1.29 | 0.97-1.73 | .0803 |
| continuous | | | | | | | | | |
| Age, yr | 1.01 | 0.95-1.08 | .7218 | 1.01 | 0.95-1.07 | .8311 | 1.01 | 0.95-1.08 | .7509 |
| Women | 0.55 | 0.23-1.33 | .1837 | 0.51 | 0.21-1.24 | .1349 | 0.55 | 0.23-1.32 | .1797 |
| Biomarker- | 2.63 | 1.10-6.32 | .0305 | 3.64 | 1.51-8.80 | .0041 | 4.25 | 1.76-10.26 | .0013 |
| categorical | | | | | | | | | |
| Age, yr | 1.02 | 0.96-1.08 | .5786 | 1.02 | 0.96-1.09 | .4909 | 1.03 | 0.97-1.09 | .4241 |
| Women | 0.59 | 0.25-1.37 | .2157 | 0.50 | 0.21-1.21 | .1242 | .047 | 0.19-1.16 | .1000 |

Cox proportional hazards models were used to assess the ability of CSF YKL-40/Aβ42, tau/Aβ42, and ptau/Aβ42 to predict (A) conversion from cognitive normalcy (CDR 0) to cognitive impairment (CDR > 0) and (B) progression from very mild dementia (CDR 0.5) to mild or moderate dementia (CDR > 0.5).
Biomarker measures were analyzed as both continuous and categorical variables, and were converted to standard Z-scores to allow comparison of hazard ratios between different biomarkers.
In evaluating risk, “Biomarker” analyses (YKL-40/Aβ42, tau/Aβ42, ptau/Aβ42) were adjusted for age and gender. Likewise, analyses for “Age” were adjusted for biomarker and gender, and analyses for “Women” were adjusted for biomarker and age.
Abbreviations:
HR, hazard ratio;
CI, confidence interval.

TABLE 8
Utility of CSF Biomarkers in Predicting Conversion from CDR 0 to CDR > 0

| | YKL-40 | | | tau | | | ptau | | | Aβ42 | | |
|---|---|---|---|---|---|---|---|---|---|---|---|---|
| | | | p | | | p | | | p | | | p |
| | HR | 95% CI | value | HR | 95% CI | value | HR | 95% CI | value | HR | 95% CI | value |
| Biomarker- | 0.95 | 0.61-1.47 | .8081 | 1.45 | 1.11-1.90 | .0072 | 1.47 | 1.14-1.91 | .0036 | 0.41 | 0.23-0.73 | .0021 |
| continuous | | | | | | | | | | | | |
| Age, yr | 1.06 | 1.01-1.12 | .0211 | 1.07 | 1.01-1.12 | .0170 | 1.07 | 1.01-1.12 | .0148 | 1.05 | 1.00-1.10 | .0672 |
| Women | .050 | 0.22-1.12 | .0919 | 0.50 | 0.22-1.12 | .0914 | 0.51 | 0.23-1.13 | .0981 | 0.50 | 0.23-1.12 | .0923 |
| Biomarker- | 1.00 | 0.42-2.33 | .9901 | 1.88 | 0.86-4.09 | .1114 | 2.69 | 1.22-5.93 | .0139 | 0.34 | 0.10-1.16 | .0841 |
| categorical | | | | | | | | | | | | |
| Age, yr | 1.06 | 1.01-1.11 | .0225 | 1.06 | 1.01-1.12 | .0211 | 1.07 | 1.01-1.13 | .0105 | 1.05 | 1.00-1.11 | .0429 |
| Women | 0.51 | 0.23-1.13 | .0968 | 0.52 | 0.24-1.16 | .1097 | 0.55 | 0.25-1.23 | .1449 | 0.49 | 0.22-1.08 | .0754 |

Cox proportional hazards models were used to assess the ability of CSF YKL-40, tau, ptau, and Aβ42 to predict conversion from cognitive normalcy (CDR 0) to cognitive impairment (CDR > 0).
Biomarker measures were analyzed as both continuous and categorical variables.
In evaluating risk, “Biomarker” analyses (YKL-40, tau, ptau, Aβ42) were adjusted for age and gender. Likewise, analyses for “Age” were adjusted for biomarker and gender, and analyses for “Women”were adjusted for biomarker and age.
Abbreviations:
HR, hazard ratio;
CI, confidence interval.

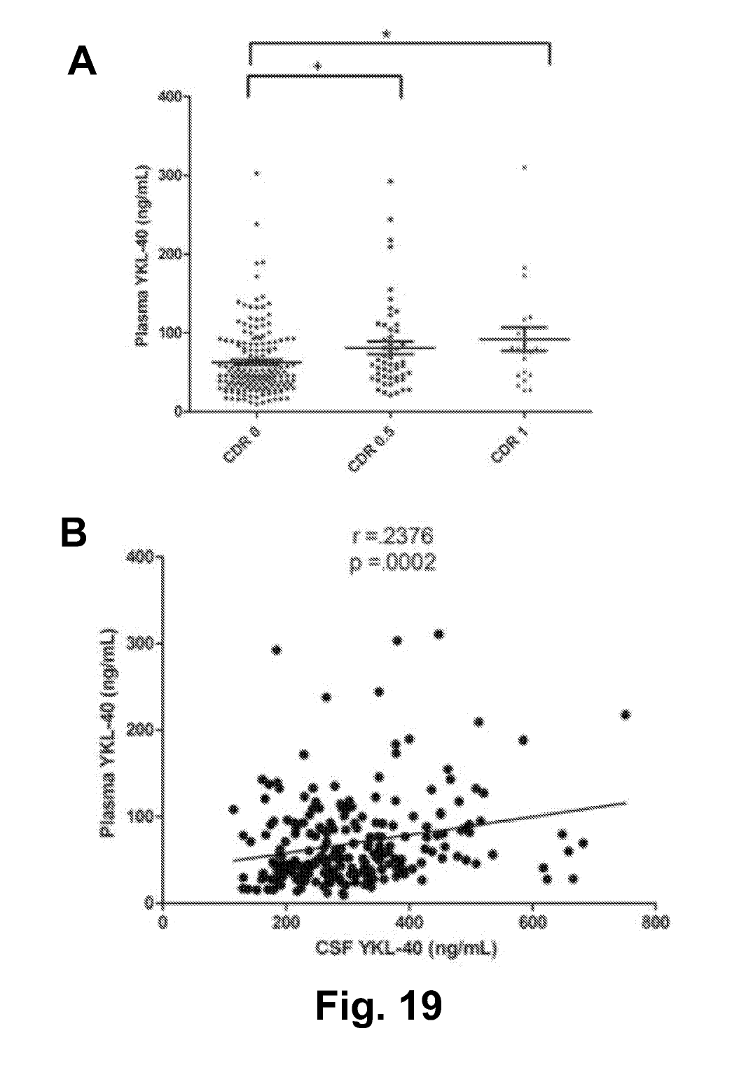

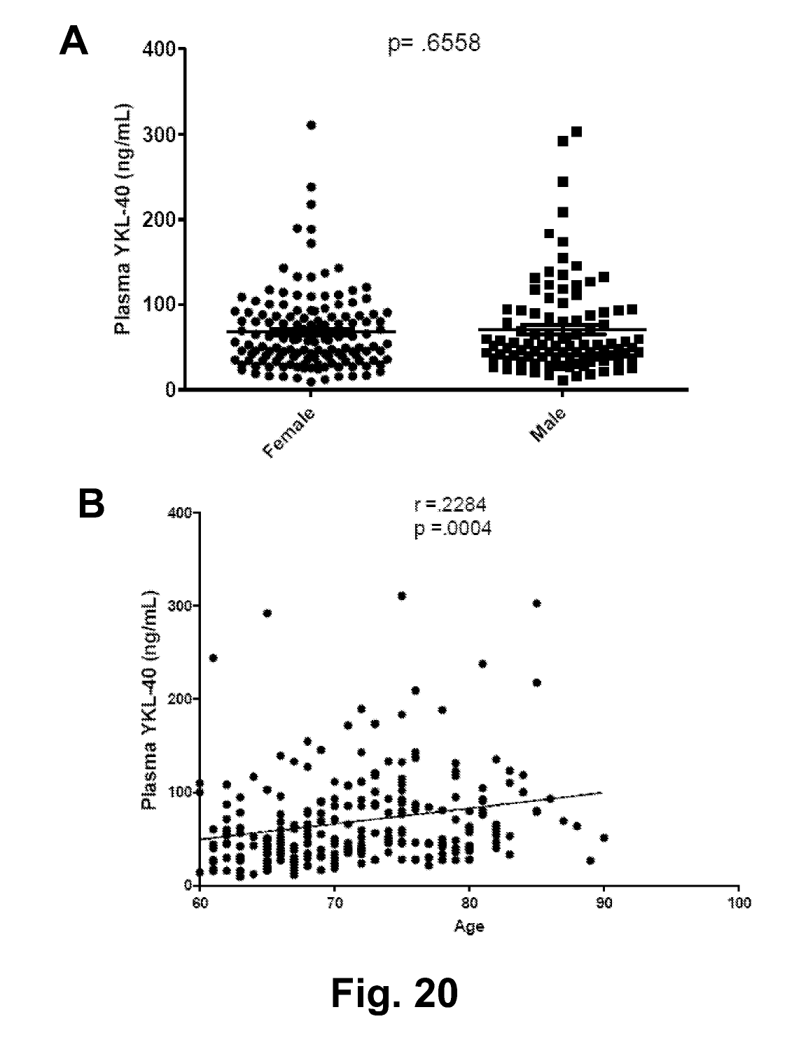

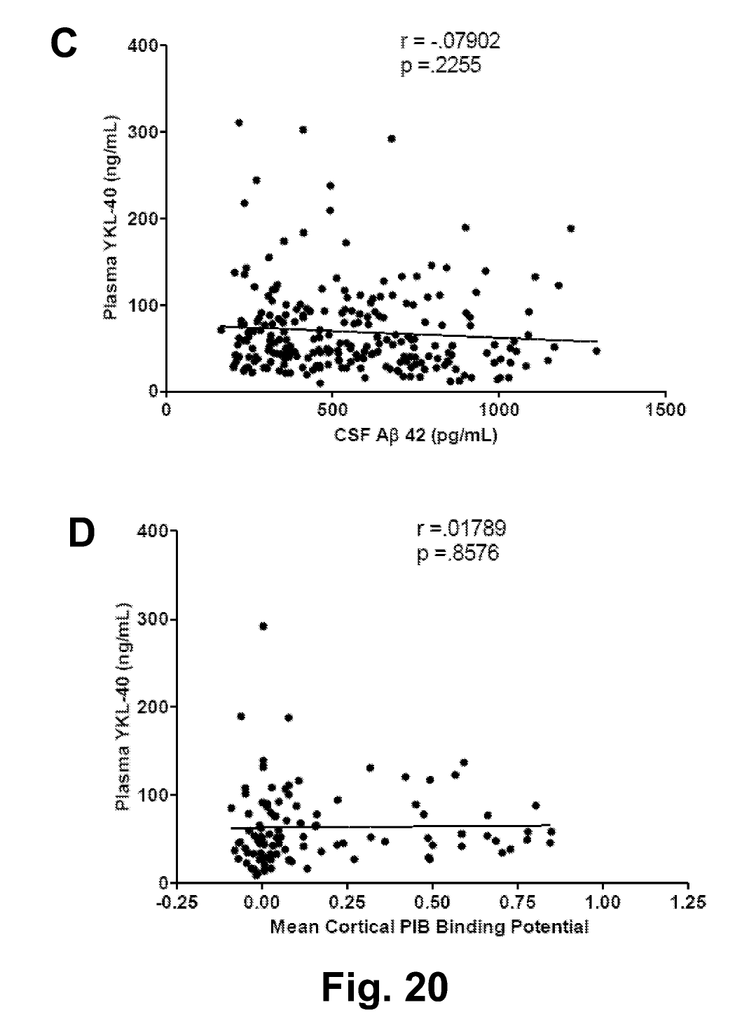

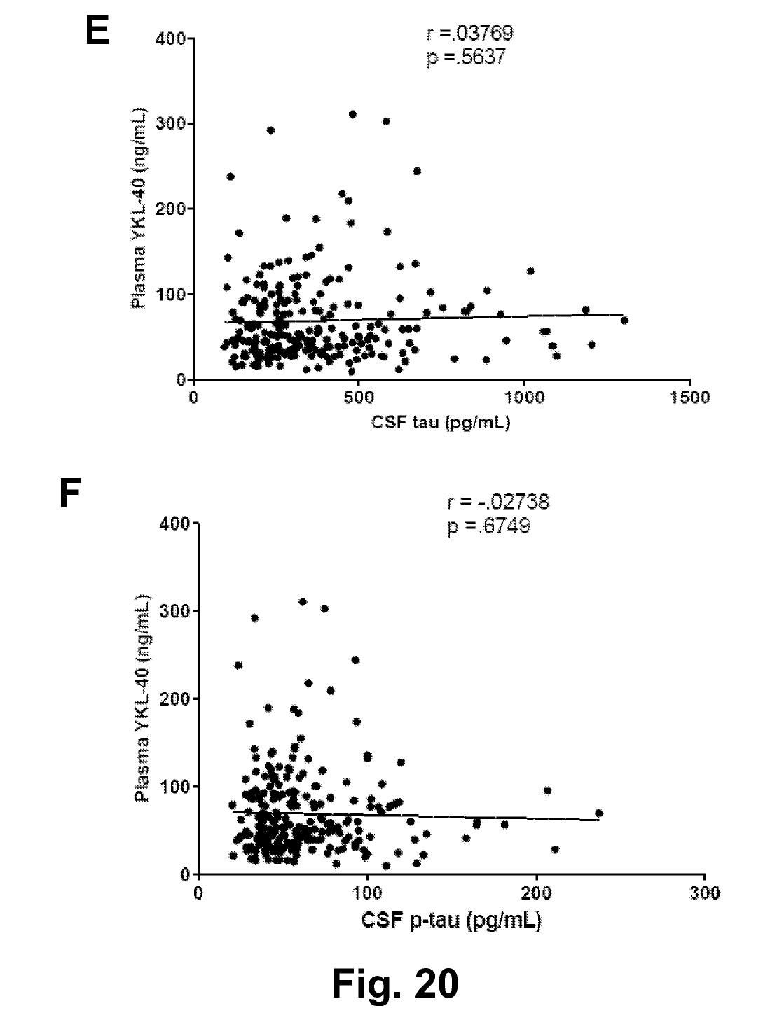

Plasma YKL-40 Demonstrates Limited Utility as AD Biomarker: To evaluate plasma YKL-40 as a potential AD biomarker, we applied the ELISA to 237 plasma samples from the validation cohort. Mean plasma YKL-40 was significantly higher in the CDR 0.5 and CDR 1 vs CDR 0 group (p=0.046, p=0.031, respectively, One-way ANOVA, Tukey posthoc), with percent increases similar to those observed in CSF (FIG. 19A). Plasma and CSF YKL-40 levels correlated modestly (r=0.2376, p=0.0002) (FIG. 19B), with levels roughly 5-fold higher in CSF. Plasma YKL-40 also correlated with increasing age (r=0.2284, p=0.0004), but not with gender (p=0.6558), CSF Aβ42 (r=−0.07902, p=0.2255), CSF tau (r=0.03769, p=0.5637), CSF ptau181 (r=−0.02738, p=0.6749), or cortical amyloid load (r=0.01789, p=0.8576) (FIG. 20). Plasma YKL-40 did not demonstrate utility for predicting cognitive decline (not shown).

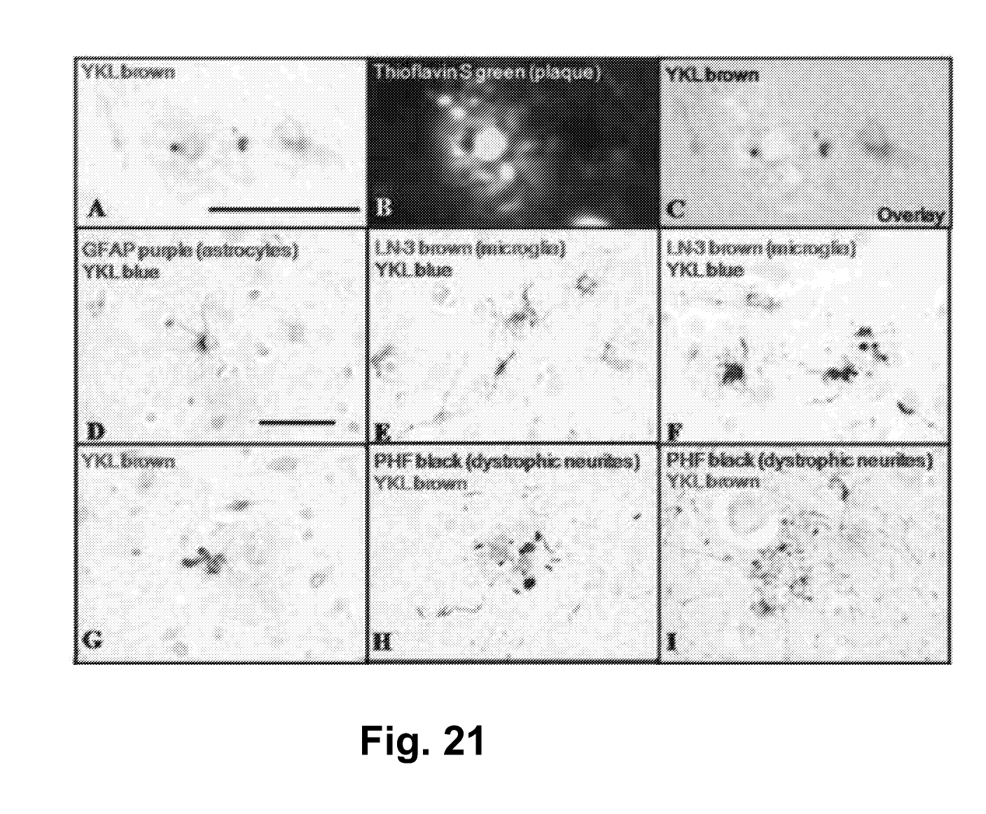

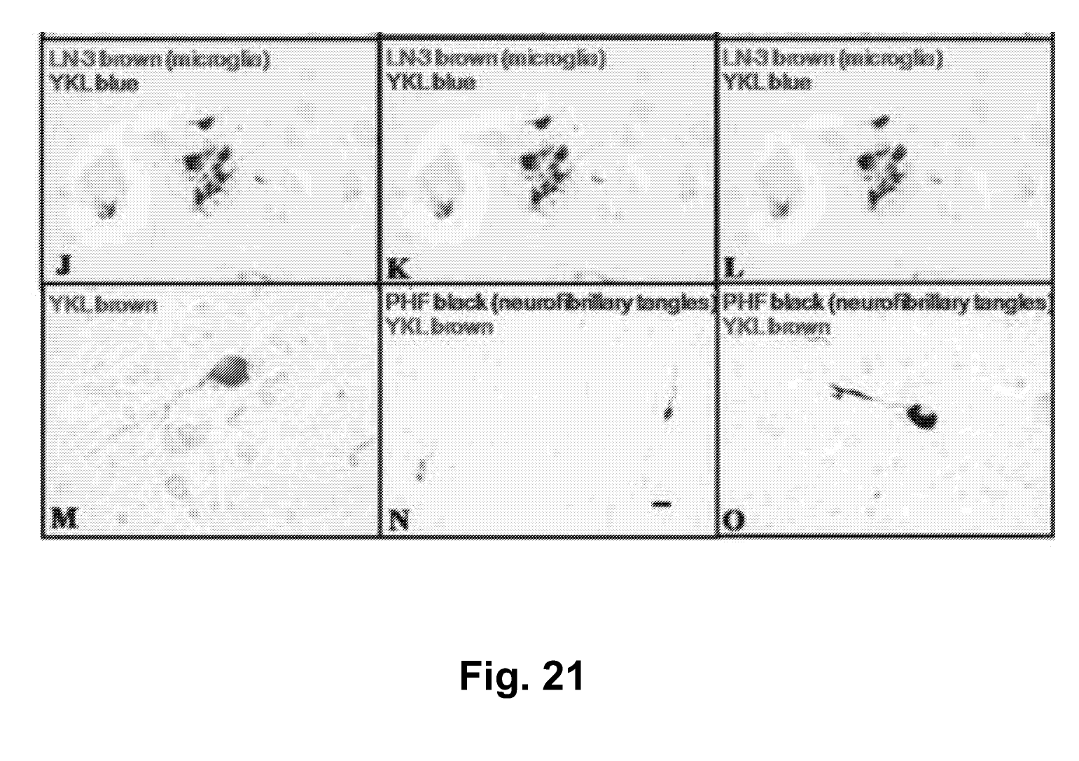

In AD Brain, YKL-40 is Expressed in Astrocytes in Vicinity of Plaques and in Rare White Matter Neurons: To investigate potential source(s) of YKL-40 in AD, we performed single and double-label immunohistochemistry on human frontal cortex. YKL-40 immunoreactivity was observed in the vicinity of a subset of thioflavin S-positive amyloid plaques (FIG. 21A,B,C) within GFAP-positive astrocytes (FIG. 8D), and not within microglia stained with LN-3 (FIG. 21E,F) or lectin RCA-1 (not shown). YKL-40 immunoreactivity was also present in plaque-associated cell processes (FIG. 21G) that lacked reactivity for dystrophic neurite marker PHF-1 (FIG. 21H) and microglial marker LN-3 (FIG. 21J,K,L representing adjacent focal planes), and that may represent astrocytic processes (suggested in FIG. 21I by the plaque-associated YKL-40-positive astrocyte in lower left quadrant). YKL-40 immunoreactivity was also observed within the superficial cortical white matter in rare neurons (FIG. 21M,N,O) with occasional PHF-1-positive neurofibrillary tangles (FIG. 21N,O). These neurons may represent cells of multiform layer VI and/or “interstitial neurons” of the white matter.

### Discussion:

This study has shown that CSF YKL-40, a novel inflammatory biomarker for AD, is increased in AD, and, together with Aβ42, can assist in prognosis of patients and clinical trial participants who are under examination for the preclinical and early clinical stages of AD.

Having identified CSF YKL-40 as a AD biomarker through non-biased proteomics, the biomarker was further verified using a commercially available ELISA, and more importantly, the verification validated the results in a much larger, independent cohort. By including very mildly impaired (CDR 0.5) individuals who may be classified at some other institutions as having MCI, or even “pre-MCI,” as some were insufficiently impaired to meet MCI criteria, this validation cohort revealed the power of CSF YKL-40 as a biomarker for very early stage AD. By including individuals with FTLD and PSP, we also demonstrated that CSF YKL-40 can be used for distinguishing AD from PSP.

By including individuals who were cognitively normal at the time of CSF collection, but subsequently developed cognitive impairment, this validation cohort also revealed the potential utility of YKL-40, coupled with Aβ42, to predict cognitive decline. It has previously been shown that ratios of CSF tau/Aβ42 and p-tau181/Aβ42 can predict conversion from cognitively normal to cognitively impaired over a 2-4 year period. Those findings were confirmed in a cohort of twice the size, and CSF YKL-40/Aβ42 has shown its predictive value comparable to that of these best current CSF measures. This finding is particularly notable because, whereas CSF tau is derived principally from neurons, YKL-40 appears to be secreted predominantly from astrocytes. To our knowledge, YKL-40 is the first astrocyte-derived marker shown to be useful in such a way. As shown in the studies, CSF YKL-40/Aβ42 also can be used for predicting progression of dementia from CDR 0.5 to CDR>0.5.

Plasma YKL-40's potential as an AD biomarker was also evaluated. While plasma YKL-40 levels displayed a pattern of elevation in the CDR 0.5 and 1 groups similar to that observed for CSF, and plasma and CSF levels were modestly correlated, plasma YKL-40 did not show similar prognostic utility. Whether this increase in plasma YKL-40 reflects passive or active export of central nervous system (CNS)-derived YKL-40 or coincident peripheral production in response to a systemic inflammatory signal is unclear. Similar coincident elevations of CSF and serum YKL-40 levels have been reported with aneurysmal subarachnoid hemorrhage and multiple sclerosis. However, in the setting of CNS infection, CSF levels of YKL-40 appear to rise without a concomitant increase in serum levels, suggesting that YKL-40 produced in the brain does not influence serum/plasma levels. Data to address the converse—whether YKL-40 produced in the periphery can influence CSF levels—have not yet been reported. This issue is important to assess in future studies because peripheral inflammatory and neoplastic conditions are not uncommon within populations most likely to be screened for AD.

To examine its role in AD and to identify potential sources of CSF YKL-40, human AD brain tissue were immunohistochemically double-labeled for YKL-40 and other cellspecific markers. YKL-40 were observed in a subset of plaque-associated astrocytes and in rare white matter neurons, which were demonstrated to be the origins of CSF YKL-40. Additionally, the pattern of expression within a subset of plaque-associated astrocytes explained the positive correlation between CSF YKL-40 and cortical amyloid load (FIG. 15); as amyloid plaque burden increases, so does the amount of plaque associated-astrocyte activation, and the amount of CSF YKL-40. This expression pattern may account for the lack of correlation between CSF YKL-40 and CSF Aβ42, and for the relatively equal levels of CSF YKL-40 between CDR 0.5 and CDR 1 groups; once plaque formation commences, which is estimated to occur ˜15 years prior to cognitive decline, CSF Aβ42 remains at a low steady state, so no correlation with YKL-40 would be expected. Likewise, amyloid burden appears close to its maximal extent once cognitive decline begins, so plaque burden and CSF YKL-40 levels might be expected to be similar in CDR 0.5 and CDR 1 groups. More importantly, these results supports that YKL-40 in the astrocytic neuroinflammatory response to fibrillar Aβ deposition that appears to play a role in AD pathogenesis.

What induces YKL-40 expression in the presence of AD pathology, and how increased YKL-40 expression may influence the disease process are unknown. It is reasonable to hypothesize that YKL-40 levels in plasma and CSF might be modulated by systemic or central inflammation. Defining the factors required to induce YKL-40 expression in astrocytes will be an important first step in understanding the role of YKL-40 in AD and, more generally, in the CNS.

Defining the targets of YKL-40 in the brain is also critically important for understanding its role in AD. In the periphery, YKL-40 can reportedly stimulate connective tissue cell growth; modulate the effects of inflammatory cytokines in fibroblasts; bind collagen and influence its fibrillogenesis; stimulate endothelial cell migration; modulate vascular smooth muscle cell adhesion and migration; support antigen-induced Th2 inflammatory responses; and stimulate alveolar macrophages to release metalloproteinases and proinflammatory and fibrogenic chemokines. In the brain, YKL-40 is reported to release extracellular matrix-bound bFGF.

This study identifies YKL-40 as a novel astrocyte-derived CSF biomarker that can distinguish groups of AD and control subjects and predict risk of developing dementia among cognitively normal subjects. Nevertheless, like all AD biomarker candidates to date, YKL-40 is likely to have less value when applied in isolation, and, alone, will be insufficient to provide definitive information for an individual patient. While significant differences in mean CSF and plasma YKL-40 levels exist between CDR 0 and CDR 0.5, and CDR 0 and CDR 1 groups, the ranges of YKL-40 values among the groups show considerable overlap. This overlap may stem from several sources. The greatest contribution is likely due to the inclusion of individuals with asymptomatic (preclinical) AD pathology in the CDR 0 group; AD neuropathology is present in ˜25% of non-demented individuals age ≧75 years. It is also possible that different alleles of the CHI3L1 gene may influence baseline or reactive levels of YKL-40 protein expression, or that members of this cohort may be afflicted by other diseases that affect CSF YKL-40 levels. For example, elevated CSF YKL-40 has been reported in the setting of other CNS pathologies; however, most of these conditions would be easily distinguishable from early AD on the basis of clinical assessment. It is important to note that the overlap observed for CSF YKL-40 is comparable to that seen for the best biomarkers identified to date, CSF Aβ42 and CSF tau (FIGS. 13D and E). The best use of YKL-40 may be in a panel of biomarkers that provide complementary information to guide diagnosis, prognosis, clinical trial design, and treatment decisions. Indeed, in other work stemming from this 2-D DIGE study, stepwise logistic regression analyses indicate that YKL-40, as part of a panel with other CSF biomarkers, contributes additional sensitivity and specificity for discriminating mildly demented individuals from cognitively normal individuals (Perrin R J, Craig-Schapiro R, Holtzman D M et al. 2010, in preparation). Additionally, YKL-40 confers specificity to a panel by distinguishing PSP or other illnesses from AD, as our early results have shown. It will be of interest in future studies to confirm these results and to evaluate CSF YKL-40 levels in the setting of additional dementing conditions. Perhaps more importantly, YKL-40, for its own part, contributes diagnostic sensitivity for early cognitive impairment, prognostic information for risk of cognitive decline in normal and very mildly impaired individuals, and, more fundamentally, a direct estimate of neuroinflammation, which tau and Aβ42 do not provide.

What is claimed is:

1. A biomarker for AD, the biomarker comprising the level of YKL-40 in a CSF sample from a subject.

2. The biomarker of claim 1, wherein the YKL-40 level is YKL-40 protein concentration.

3. A biomarker for AD, the biomarker comprising the ratio of CSF YKL-40/Aβ42 in a CSF sample from a subject.

4. The biomarker of claim 3, wherein the YKL-40 level is YKL-40 protein concentration.

5. A method for detecting or monitoring AD, the method comprising:
a. quantifying the level of YKL-40 in a bodily fluid of the subject, and
b. determining if the quantified level of YKL-40 is elevated in comparison to the average YKL-40 level for a subject with a CDR of 0.

6. The method of claim 5, wherein an elevated YKL-40 level in comparison to the average YKL-40 level indicates a diagnosis of AD.

7. The method of claim 5, wherein an elevated YKL-40 level in comparison to the average YKL-40 level indicates a prognosis of AD.

8. The method of claim 5, wherein the YKL-40 level measured is YKL-40 protein concentration.

9. The method of claim 8, further comprising measuring YKL-40 protein concentration by ELISA.

10. The method of claim 5, wherein the subject is at risk for AD.

11. The method of claim 5, wherein the subject has no clinical signs of AD.

12. The method of claim 5, wherein the subject is human.

13. The method of claim 5, wherein the bodily fluid is CSF.

14. The method of claim 5, further comprising
a. quantifying the level of at least one other AD biomarker in a bodily fluid of the subject, and
b. determining if the quantified level of the additional AD biomarker or biomarkers is elevated or depressed in comparison to the average level for that biomarker for a subject with a CDR of 0.

15. The method of claim 14, further comprising quantifying three or more biomarkers.

16. The method of claim 14, further comprising quantifying five or more biomarkers.

17. The method of claim 14, further comprising quantifying ten or more biomarkers.

18. The method of claim 14, further comprising selecting the additional biomarkers from the group comprising ACT, ZAG, CNDP1, ATIII, Aβ42, tau, YKL-40/Aβ42, tau/Aβ42, and p-tau181/Aβ42.

19. The method of claim 5, where the subject's response to AD treatment is monitored.
